(19) United States
(12) Patent Application Publication
(10) Pub. No.: US 2019/0105436 A1
(43) Pub. Date: Apr. 11, 2019

(54) BLOOD PUMP AND METHOD OF MANUFACTURING BLOOD PUMP

(71) Applicant: SUN MEDICAL TECHNOLOGY RESEARCH CORPORATION, Suwa-shi (JP)

(72) Inventor: Masami UCHIDA, Nagano (JP)

(21) Appl. No.: 16/154,728

(22) Filed: Oct. 9, 2018

(30) Foreign Application Priority Data
Oct. 10, 2017 (JP) 2017-196796

(51) Int. Cl. A61M 1/10

(52) U.S. Cl. CPC A61M 1/1025; A61M 1/12; A61M 1/1005; A61M 1/1086

(57) ABSTRACT

A blood pump includes: a base body; a casing fitted on the base body; a blood supply mechanism housed in a pump chamber surrounded by the base body and the casing; and a drive element for supplying energy to the blood supply mechanism. The base body has: an approximately horizontal surface including a first contact surface which is brought into contact with a second contact surface; a first engaging portion; and a base body stepped portion. The casing has: a second engaging portion; and a casing edge portion. The second engaging portion engages with the first engaging portion. A welded mark which connects the casing edge portion and the base body stepped portion to each other is formed at least at two portions which are separated from each other. The second contact surface of the casing is pressed so as to be brought into contact with the first contact surface.

### BACKGROUND OF THE INVENTION

### 1. Field of the Invention

The present invention relates to a blood pump and a method of manufacturing a blood pump.

### 2. Description of the Related Art

There has been known a blood pump which is used in an auxiliary artificial heart or the like (see JP-A-2009-297174, for example). The blood pump is provided for assisting a blood supply function of the user's heart. The blood pump has, as a basic function thereof, a blood supply function for supplying blood of the user into the inside of a body of the user by allowing the blood of the user to flow into a pump chamber and to flow out from the pump chamber. In the inside of the pump chamber of the blood pump, it is necessary to house a blood supply mechanism such as an impeller which forms a movable part. Accordingly, in general, in assembling the blood pump, a base body and a casing are prepared as separate parts respectively, and a blood supply mechanism is housed in a pump chamber surrounded by the base body and the casing and, thereafter, the blood pump is completed by combining the base body and the casing together.

FIG. 22 and FIG. 23 are views for describing a conventional blood pump 800. FIG. 22 is an exploded perspective view showing a state before a casing 820 is combined with a base body 810. FIG. 23 is a cross-sectional view showing a state after the casing 820 is combined with the base body 110.

As shown in FIG. 22 and FIG. 23, the conventional blood pump 800 includes: the base body 810; the casing 820 fitted on the base body 810; a blood supply mechanism 830 housed in a pump chamber 850 surrounded by the base body 810 and the casing 820; and a drive element 840 mounted on the base body 810 and supplying energy to the blood supply mechanism 830. In such a blood pump 800, the blood supply mechanism 830 allows blood to flow into the pump chamber 850 and to flow out from the pump chamber 850, and supplies blood into the inside of a body of a user (not shown in the drawing). In the description of the conventional blood pump 800, assume a direction that the casing 820 is fitted on the base body 810 by sliding as a z direction, a direction perpendicular to the z direction as an x direction, a direction perpendicular to the z direction and the x direction respectively as a y direction, and a direction directed from a center portion of the base body 810 to the outside of the base body 810 when an xy plane is viewed in a plan view along the z direction as an r direction. The base body 810 has: a blood contact surface 812 which faces the pump chamber 850; a first contact surface 813 which is brought into contact with the casing 820; and a screw fastening margin 814 formed on a more r direction side than the first contact surface 813; and female screws 815 formed in a region of the screw fastening margin 814 in a state where the female screws 815 extend in the z direction. On the other hand, the casing 820 has: a second contact surface 822 (not shown in FIG. 22) formed on the casing 820 at a position corresponding to the first contact surface 813; and play holes 825 formed on an edge side of the casing 820. Then, in the blood pump 800, by allowing male screws 890 to pass through the play holes 825 formed in the casing 820 and to threadedly engage with the female screws 815 of the base body 810, the base body 810 and the casing 820 are combined together and are threaded with each other, and the second contact surface 822 of the casing 820 is pressed so as to be brought into contact with the first contact surface 813 of the base body 810.

In the conventional blood pump 800, the base body 810 and the casing 820 are formed as separate parts from each other and hence, the blood supply mechanism 830 which forms the movable part can be housed in the pump chamber. Further, the male screws 890 pass through the play holes 825 formed in the casing 820 and threadedly engage with the female screws 815 of the base body 810 and hence, the base body 810 and the casing 820 are combined together and are threaded with each other, and the second contact surface 822 of the casing 820 is pressed so as to be brought into contact with the first contact surface 813 of the base body 810. Accordingly, even when pressure in the pump chamber 850 is increased, it is possible to prevent the formation of a gap between the second contact surface 822 on a casing 820 side and the first contact surface 813 on a base body 810 side. In other words, it is possible to prevent the second contact surface 822 from lifting from the first contact surface 813.

For a reference purpose, one required specification of the blood pump is described. That is, such a required specification is that even when a pressure in the pump chamber on a blood side is increased, the formation of a gap between the second contact surface on a casing side which defines the pump chamber and the first contact surface on a base body side which defines the pump chamber must be prevented by all means.

Assume a case where a gap is formed between the second contact surface and the first contact surface. Due to the formation of such a gap, a slight amount of blood which intrudes into the gap is congested and is solidified. There is a possibility that this solidified blood clot (thrombus) is peeled off from the gap due to agitation in the pump chamber by a blood supply mechanism such as an impeller and returns to the pump chamber again. Depending on a case, there is also a possibility that such a blood clot flows out into the inside of a body of a user (patient) and causes an undesirable condition. Due to such a reason, the formation of the gap between the second contact surface and the first contact surface must be prevented by all means. Even when a pressure in the pump chamber is increased, to prevent the formation of a gap between the second contact surface on a casing side and the first contact surface on a base body side, it is necessary to constantly apply a pressing force larger than a force pressed from a pump chamber side (hereinafter, also simply referred to as “pressing force”) from the second contact surface on a casing side to the first contact surface on a base body side.

It is considered that a strength of a force which acts so as to prevent peeling off of the casing from the base body (referred to as “casing peel-off strength” for the convenience sake in this specification) is also relevant to the above-mentioned “pressing force” and hence, a correlation substantially exists between the casing peel-off strength and the pressing force. In the same manner as the pressing force, it is necessary for the blood pump to sufficiently ensure the casing peel-off strength so as to prevent the casing from being peeled off from the base body (to prevent the casing from lifting from the base body) even when a pressure in the pump chamber is increased.

### SUMMARY OF INVENTION

However, it is necessary for the conventional blood pump 800 to adopt the screws (the male screws 890 and the female screws 815) having a relatively large size to maintain a pressing force and to ensure a casing peel-off strength. Accordingly, as a width of the screw fastening margin 814 (the width of the screw fastening margin 814 being indicated by MG1 in FIG. 22 and FIG. 23), it is necessary to ensure a relatively large width. As a result, it is unavoidable that a diameter, a volume and the like of the whole blood pump become relatively large.

On the other hand, in a medical field, there has been a strong demand for the miniaturization of a blood pump. When the blood pump is small, it is possible to embed the blood pump into the inside of the body of a person having a small physical build such as a child, for example (a person of a physical build with not so large of a chest) and hence, the number of people who can use a blood pump can be increased.

The present invention has been made in view of the above-mentioned circumstances, and it is an object of the present invention to provide a blood pump which can increase a casing peel-off strength compared to conventional blood pumps while maintaining a pressing force from a second contact surface on a casing side to a first contact surface on a base body side, and is smaller in size than conventional blood pumps. It is another object of the present invention to provide a method of manufacturing such a blood pump.

[1] A blood pump according to the present invention includes:

a base body;

a casing fitted on the base body;

a blood supply mechanism housed in a pump chamber surrounded by the base body and the casing; and

a drive element mounted on the base body for supplying energy to the blood supply mechanism, wherein

the blood supply mechanism is configured to allow blood to flow into the pump chamber and to flow out from the pump chamber so as to supply blood into the inside of a body of a user, wherein

assuming a direction that the casing is fitted on the base body by sliding as a z direction, a direction perpendicular to the z direction as an x direction, a direction perpendicular to the z direction and the x direction respectively as a y direction, a direction directed from a center portion of the base body to the outside of the base body when an xy plane is viewed in a plan view along the z direction as an r direction, a direction opposite to the z direction as a −z direction, and a direction opposite to the r direction as a −r direction,

the base body has:

an approximately horizontal surface formed on a −z direction side, and including a blood contact surface which faces the pump chamber and a first contact surface which is brought into contact with the casing;

a first engaging portion formed on an r direction side of the base body; and

a base body stepped portion formed on a z direction side of the first engaging portion,

the casing has:

a casing body having a second contact surface at a position which corresponds to the first contact surface;

a second engaging portion formed at a position of an edge side of the casing with respect to the casing body;

a casing intermediate portion positioned between the casing body and the second engaging portion; and

a casing edge portion positioned on a side opposite to a side of the casing intermediate portion with respect to the second engaging portion,

the base body stepped portion is disposed at a position which corresponds to the casing edge portion in a state where the casing is fitted on the base body, and

the blood pump is configured such that the second engaging portion of the casing engages with the first engaging portion of the base body, a welded mark which connects the casing edge portion and the base body stepped portion to each other is formed at least at two portions which are separated from each other, and the second contact surface of the casing is pressed so as to be brought into contact with the first contact surface of the base body.

In the blood pump according to the present invention, the welded mark which connects the casing edge portion and the base body stepped portion to each other is formed at least at two portions, and the second contact surface of the casing is pressed so as to be brought into contact with the first contact surface of the base body. That is, the casing and the base body are strongly joined to each other by welding at least at two portions in addition to joining of the casing and the base body obtained by engagement between the first engaging portion and the second engaging portion. Accordingly, while maintaining a pressing force on contact surfaces (hereinafter, the first contact surface and the second contact surface being collectively simply referred to as “contact surfaces”) in the same manner as the conventional blood pump, the blood pump according to the present invention can increase a casing peel-off strength compared to the conventional blood pump. Further, the blood pump according to the present invention adopts the structure which requires no screws and hence, it is possible to provide a miniaturized blood pump compared to a conventional blood pump.

Accordingly, it is possible to provide the blood pump which can increase a casing peel-off strength compared to conventional blood pumps while maintaining a pressing force from the second contact surface on a casing side to the first contact surface on a base body side, and is smaller in size than conventional blood pumps.

[2] In the blood pump according to the present invention, it is preferable that,

on the casing edge portion, a casing edge portion lower surface which is a surface of an edge of the casing on a z direction side, a casing edge portion outer surface which is a surface of the edge of the casing on an r direction side, and a casing outer end which is a corner formed by the casing edge portion lower surface and the casing edge portion outer surface be formed,

on the base body stepped portion, a base body stepped portion upper surface which is formed in an abutting manner with the casing edge portion lower surface in a state where the casing is fitted on the base body, a base body stepped portion outer surface which is a surface of the base body stepped portion on an r direction side, and a base body outer end which is a corner formed by the base body stepped portion upper surface and the base body stepped portion outer surface be formed, and

in the blood pump, the welded mark which connects the casing edge portion and the base body stepped potion to each other be formed at least at two portions which are separated from each other between a first profile of the base body outer end formed so as to surround a rotary axis of the blood supply mechanism and a second profile of the casing outer end formed so as to surround the rotary axis of the blood supply mechanism.

[3] In the blood pump according to the present invention, it is preferable that

a first tapered portion inclined toward the −r direction as the first tapered portion extends from a tapered outer end portion in the z direction be formed on the first engaging portion, and

a second tapered portion inclined toward the r direction as the second tapered portion extends from a tapered inner end portion in the −z direction on an inner wall of the casing be formed on the second engaging portion.

Due to the engagement between the first tapered portion (base body side) of the first engaging portion and the second tapered portion (casing side) of the second engaging portion, a force in a direction which presses the casing in the z direction (base body side) is generated. Such a force which presses the casing in the z direction can be further added as a portion of a pressing force applied to the contact surfaces, and such an additional force also contributes to the enhancement of a casing peel-off strength. In this manner, according to the present invention, a casing peel-off strength can be further enhanced compared to conventional blood pumps.

[4] In the blood pump according to the present invention, it is preferable that

assuming an inner diameter of the tapered inner end portion of the second tapered portion of the casing as ϕA and an outer diameter of the tapered outer end portion of the first tapered portion of the base body as ϕB before the casing is fitted on the base body, a relationship of ϕA<ϕB be established, and

the casing be fitted on the base body in a state that a position of the tapered inner end portion of the second tapered portion is disposed at a portion of the first tapered portion shifted in a z direction side from the tapered outer end portion, the portion being a portion of the first tapered portion away a first tapered terminal end portion on a side opposite to the tapered outer end portion.

[5] In the blood pump according to the present invention, it is preferable that

assuming an angle made by a profile of an inclined surface of the first tapered portion and the z direction as ϕ1 and an angle made by a profile of an inclined surface of the second tapered portion and the z direction as ϕ2, a relationship of 0≤ϕ1≤ϕ2 be established.

[6] In the blood pump according to the present invention, it is preferable that

assuming a thickness of the casing body in a direction perpendicular to a tangent plane on an outer side of the casing body as t1, a thickness of the casing intermediate portion in a direction perpendicular to a tangent plane of an outer side of the casing intermediate portion as t2, and a thickness of the second engaging portion in a direction perpendicular to a tangent plane of an outer side of the second engaging portion as t3, a relationship of t1>t2>t3 be established, and

with respect to an inclined surface which forms the second tapered portion of the second engaging portion, on a second tapered terminal end portion side which is a side opposite to the tapered inner end portion, the casing have a smallest thickness.

[7] In the blood pump according to the present invention, it is preferable that

a width of the welded mark in the z direction fall within a range of from 0.3 mm to 2.0 mm, and

a total length of the welded marks fall within a range of from 2 mm to 40 mm when the welded marks are viewed along the z direction.

[8] In the blood pump described in the above-mentioned [7], it is preferable that

the total length of the welded marks be 5 mm or more when the welded marks are viewed along the z direction.

[9] In the blood pump described in the above-mentioned [7] or [8], it is preferable that

the total length of the welded marks be 30 mm or less when the welded marks are viewed along the z direction.

[10] In the blood pump according to the present invention, it is preferable that

an underfill be formed on the welded mark, and

a gap between a deepest portion of the underfill and the casing edge portion outer surface or the base body stepped portion outer surface fall within a range of from 0.05 mm to 0.3 mm.

[11] In the blood pump according to the present invention, it is preferable that

assuming a thickness of the casing intermediate portion in a direction perpendicular to the tangent plane of the outer side of the casing intermediate portion which is a thickness of a portion of the casing having a smallest thickness as t2′, and

assuming a thickness of the second engaging portion in a direction perpendicular to the tangent plane of the outer side of the second engaging portion as t3, a relationship of t2′<t3 be established.

[12] In the blood pump according to the present invention, it is preferable that

the base body have a packing groove in which a packing is disposed at a position on a more −z direction side of the first engaging portion,

the casing have an intermediate portion inner wall at a position of the casing intermediate portion, and

the packing be disposed such that the packing is sandwiched between the packing groove and the intermediate portion inner wall.

[13] A method of manufacturing a blood pump according to the present invention is a method of manufacturing a blood pump which manufactures the blood pump described in any one of the above-mentioned [1] to [12], the method includes in a following order:

a sub unit preparation step for preparing the base body, the casing, and the blood supply mechanism;

a blood supply mechanism mounting step for mounting the blood supply mechanism on the base body;

a casing fitting step for fitting the casing on the base body such that the second engaging portion engages with the first engaging portion by sliding of the casing in the z direction with respect to the base body; and

a welding step for performing welding so as to connect the casing edge portion and the base body stepped portion to each other at least at two portions which are separated from each other between the first profile of the base body outer end and the second profile of the casing outer end while pressing the casing to the base body in the z direction.

[14] The method of manufacturing a blood pump according to the present invention is, in the method of manufacturing a blood pump described in the above-mentioned [13], further includes in a following order after the welding step:

a blood pump disassembling step including: a welded mark separation step for separating a portion of the casing or/and the base body including a welded mark from other portions which include no welded mark; and a casing removing step for removing the casing from the base body by sliding the casing in the −z direction with respect to the base body;

a blood pump analyzing step for analyzing the blood pump;

a casing refitting step for fitting the casing on the base body again such that the second engaging portion engages with the first engaging portion by sliding the casing in the z direction with respect to the base body; and

rewelding step for performing welding again for connecting the casing edge portion and the base body stepped portion to each other at least at two portions which are separated from each other and are different from the portions where welding is performed in the welding step while pressing the casing to the base body in the z direction.

“analysis” includes the observation of a state of the blood pump, measurement of sizes of parts, an analysis of adhered materials and the like. However, “analysis” is not limited to such operations, and also includes working, readjustment and the like applied to constitutional parts of the blood pump.

### BRIEF DESCRIPTION OF DRAWINGS

FIG. 1 is a view for describing a blood pump 100 according to an embodiment 1;

FIG. 2 is a view for describing the blood pump 100 according to the embodiment 1;

FIG. 3A to FIG. 3C are views for describing a main part of the blood pump 100 according to the embodiment 1;

FIG. 4A to FIG. 4D are views for describing an abutting profile 175 which is a candidate for a portion where welding is performed in the embodiment 1;

FIG. 5A and FIG. 5B are views for describing portions where a welded mark is disposed and a length l of the welded mark 170 in the blood pump 100 according to the embodiment 1;

FIG. 6A and FIG. 6B are views for describing the welded mark 170 in the embodiment 1;

FIG. 7 is a view for describing a manner in which a pressing force F2 which acts from a second contact surface 122 to a first contact surface 113 is generated in the blood pump 100 according to the embodiment 1;

FIG. 8 is a view for describing a size relationship of the main part of the blood pump 100 according to the embodiment 1;

FIG. 9 is a flowchart for describing a method of manufacturing a blood pump according to the embodiment 1;

FIG. 10A and FIG. 10B are views for describing a welding step S40 in the embodiment 1;

FIG. 11A to FIG. 11D are views for describing a blood pump disassembling step S50 and a rewelding step S80 in the embodiment 1;

FIG. 12 is a cross-sectional view for describing a main part of a blood pump 102 according to an embodiment 2;

FIG. 13 is a cross-sectional view for describing a main part of a blood pump 103 according to an embodiment 3;

FIG. 14A and FIG. 14B are cross-sectional views for describing a main part of a blood pump 104 according to an embodiment 4;

FIG. 15 is a schematic view for describing an auxiliary artificial heart system 300 according to an embodiment 5;

FIG. 16A and FIG. 16B are views for describing a result of evaluation of a blood pump according to a provisional test example;

FIG. 17 is a view for describing a result of evaluation of a blood pump according to a test example;

FIG. 18 is a view for describing a blood pump 105 according to a modification 1;

FIG. 19 is a view for describing a blood pump 106 according to a modification 2;

FIG. 20 is a view for describing a blood pump 107 according to a modification 3;

FIG. 21A to FIG. 21D are views for describing a blood pump 108 according to a modification 4;

FIG. 22 is a view for describing a conventional blood pump 800; and

FIG. 23 is a view for describing the conventional blood pump 800.

### DESCRIPTION OF PREFERRED EMBODIMENTS

Hereinafter, a blood pump controller according to the present invention is described based on embodiments shown in drawings. The respective drawings are schematic drawings, and do not necessarily strictly reflect actual sizes.

### Embodiment 1

### 1. Basic Configuration (Structure) of Blood Pump 100 According to Embodiment 1

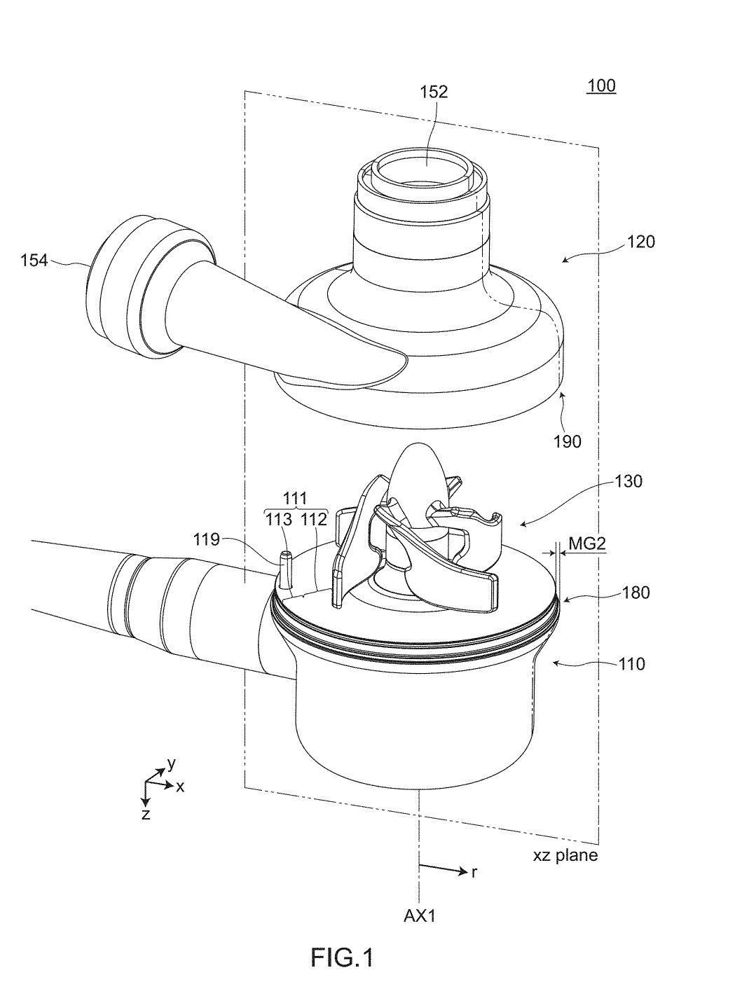

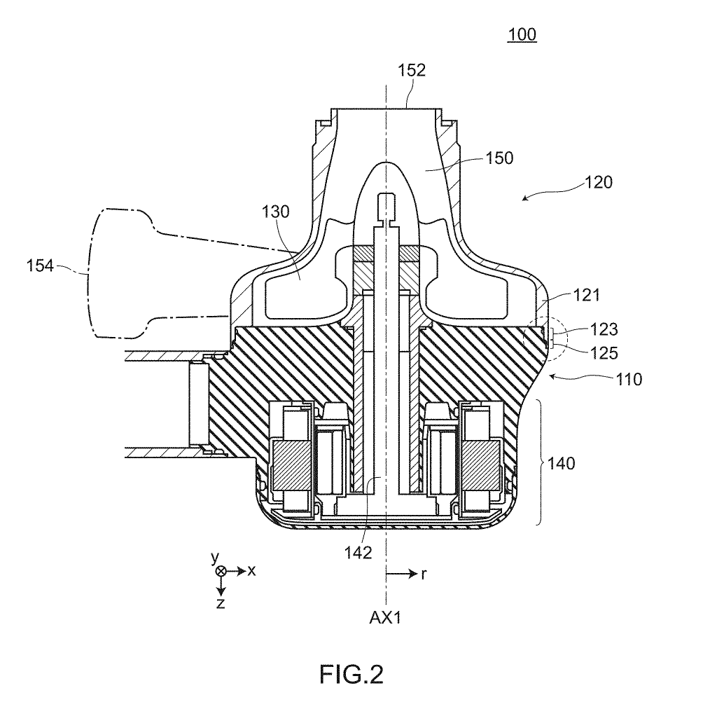

FIG. 1 and FIG. 2 are views for describing the blood pump 100 according to the embodiment 1.

FIG. 1 is an exploded perspective view of the blood pump 100 before a casing 120 is fitted on a base body 110. FIG. 2 is a cross-sectional view where a cross section of the blood pump 100 taken along an xz plane shown in FIG. 1 is viewed along a y direction, and shows a state where the casing 120 is fitted on the base body 110.

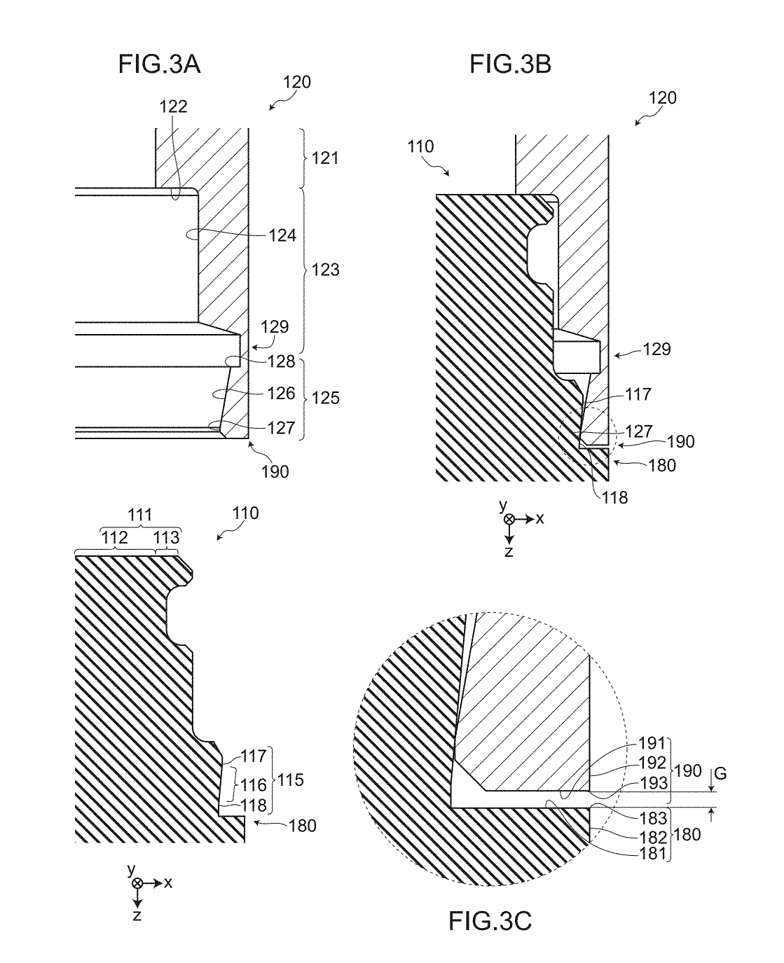

FIG. 3A to FIG. 3C are views for describing a main part of the blood pump 100 according to the embodiment 1. FIG. 3A and FIG. 3B are views where an engaging portion surrounded by a dotted line in the cross-sectional view of FIG. 2 is shown in an enlarged manner. FIG. 3A is a view showing a state before the casing 120 is fitted on the base body 110, and FIG. 3B is a view showing a state after the casing 120 is fitted on the base body 110 and before welding is performed. FIG. 3C is a view where a portion surrounded by a dotted line in FIG. 3B is further enlarged.

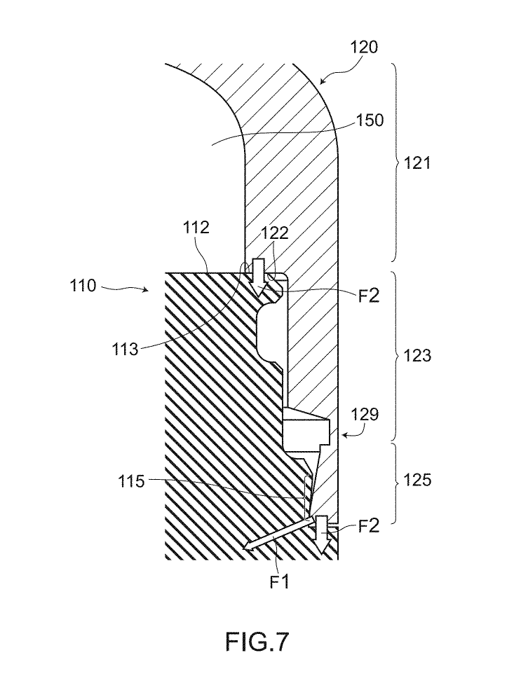

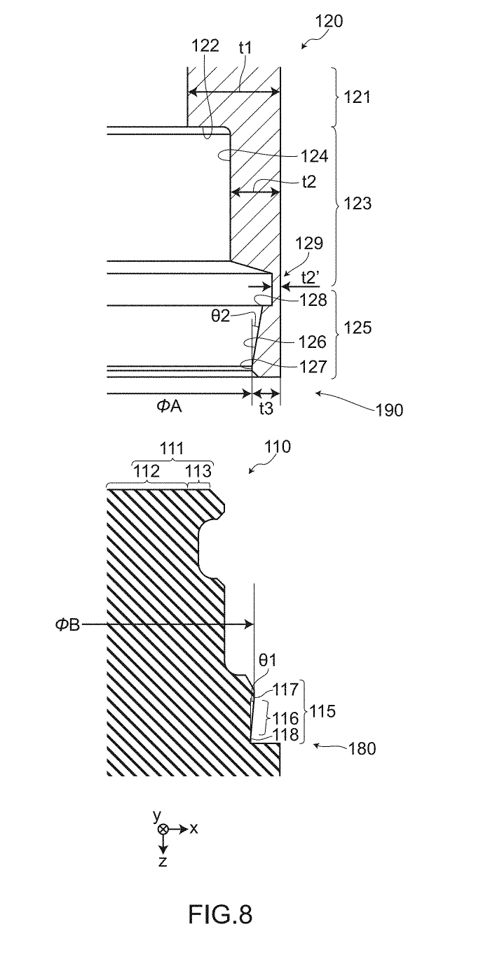

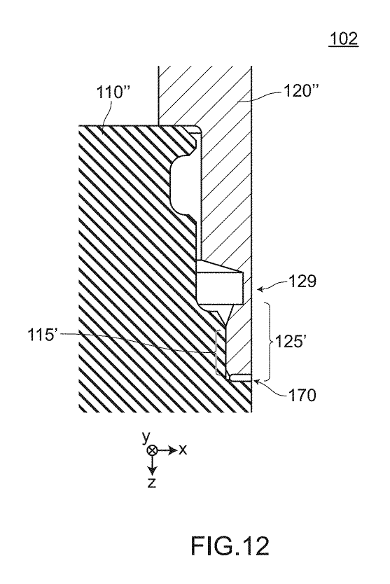

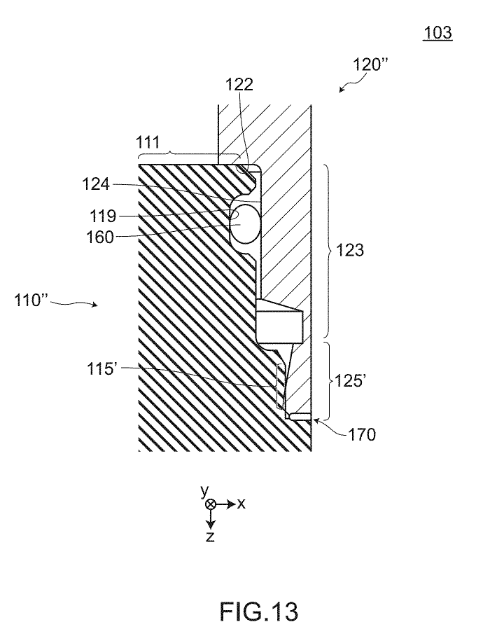

In FIG. 3A and FIG. 3B, a solid line extending in a −x direction from a hatched portion of the casing 120 expresses a shape of the casing 120 on an inner wall side (not a cut end of a cross section but a shape of the inside of the casing 120). Hereinafter, the same understanding is adopted by the configurations shown in FIG. 7, FIG. 8, FIG. 12, and FIG. 13.

### (1) Overall Structure of Blood Pump 100

The blood pump 100 has a function of allowing blood to flow into a pump chamber 150 and to flow out from the pump chamber 150 by a blood supply mechanism 130 and supplying blood into the inside of the body of a user.

The blood pump 100 according to the embodiment 1 includes: the base body 110; the casing 120 fitted on the base body 110; the blood supply mechanism 130 housed in the pump chamber 150 surrounded by the base body 110 and the casing 120 (that is, the pump chamber 150 being formed by the base body 110 and the casing 120); and a drive element 140 mounted on the base body 110 and supplying energy to the blood supply mechanism 130 (see FIG. 1 and FIG. 2).

In the description made hereinafter, assume a direction that the casing 120 is fitted on the base body 110 by sliding as a z direction, a direction perpendicular to the z direction as an x direction, a direction perpendicular to the z direction and the x direction respectively as a y direction, a direction directed from a center portion of the base body 110 to the outside of the base body 110 when an xy plane is viewed in a plan view along the z direction as an r direction. Further, assume a direction opposite to the z direction as a −z direction, and a direction opposite to the r direction as a −r direction. Still further, to facilitate the understanding of the blood pump 100, the z direction is referred to as “down”, the −z direction is referred to as “up”, the −r direction is referred to as “inside”, the r direction is referred to as “radial direction” or “outside”. With respect to the casing 120, a pump chamber 150 side of the casing 120 is referred to as “inside”, a side opposite to the pump chamber 150 is referred to as “outside”, and a direction parallel to the xy plane is referred to as “horizontal” or the like.

### (2) Blood Supply Mechanism 130 and Drive Element 140

The blood supply mechanism 130 is a movable mechanism capable of flowing and supplying blood. The blood supply mechanism 130 allows blood to flow into the pump chamber 150 and to flow out from the pump chamber 150, and supplies blood to the inside of the body of a user. The blood supply mechanism 130 is disposed in the inside of the pump chamber 150 surrounded by the base body 110 and the casing 120 (see FIG. 1 and FIG. 2).

The drive element 140 is mounted on the base body 110 and supplies energy for allowing the blood supply mechanism 130 to perform the supply of blood to the blood supply mechanism 130.

In the embodiment 1, an impeller can be adopted as the blood supply mechanism 130. A motor for driving the impeller can be adopted as the drive element 140. The impeller easily exhibits a blood supply function, and a control technique which exhibits linearity, responsiveness and the like is relatively established with respect to the motor so that the motor can relatively easily perform a control in accordance with a purpose. By driving the impeller using the motor, the blood supply mechanism 130 can exhibit an intended blood supply function efficiently thus providing a blood pump having high performance and high accuracy.

In the embodiment 1, the motor is mounted on the base body 110 such that a stator part (not shown in the drawing) and a rotor part (not shown in the drawing) of the motor are housed in the base body 110 on a z direction (down) side as viewed from an approximately horizontal surface 111 of the base body 110. A shaft 142 of the motor projects from a base body 110 side toward a −z direction (up) side. The impeller is joined to this projecting shaft 142.

The rotary axis of the blood supply mechanism 130 (impeller) is indicated by symbol AX1 (see FIG. 1 and FIG. 2).

### (3) Base Body 110

As shown in FIG. 3A to FIG. 3C (also see FIG. 1 and FIG. 2 together with the above-mentioned drawings), the base body 110 has an approximately horizontal surface 111 which is disposed in a −z direction side (upper side) and includes a blood contact surface 112 which faces the pump chamber 150 and a first contact surface 113 which is brought into contact with the casing 120.

The blood contact surface 112 is a surface with which blood of a user is brought into contact, and defines the pump chamber 150 together with the casing body 121. The first contact surface 113 is a surface which is brought into contact with a second contact surface 122 of the casing body 121 and receives an action of a pressing force from the second contact surface 122. These blood contact surface 112 and the first contact surface 113 are continuously formed, and form an approximately horizontal surface 111 as a whole.

In addition to such a configuration, the base body 110 has a first engaging portion 115 formed on an r direction side of the base body 110. The first engaging portion 115 engages with a portion of the casing 120 (a second engaging portion 125 described later in the embodiment 1).

Further, the base body 110 has a base body stepped portion 180 formed on a z direction side (lower side) of the first engaging portion 115. In other words, the base body stepped portion 180 is formed on the base body 110 (see FIG. 3A and FIG. 3B).

The base body stepped portion 180 is, in general, a portion where a direction of a tangent of a surface largely changes (typically a portion where a step is formed) when a surface of the base body 110 is traced in a z direction or in a −z direction on a cross section obtained by cutting the base body 110 by a plane which includes a z axis parallel to the z axis.

The base body stepped portion 180 is disposed and formed at a position which corresponds to a casing edge portion 190 (described in detail later) when the casing 120 is fitted on the base body 110.

The base body 110 may be formed using any material provided that the base body 110 forms a part of the blood pump 100 according to the present invention. The base body 110 may be formed using the same material as the material for forming the casing 120. However, the base body 110 (particularly in the vicinity of the first engaging portion 115 and in the vicinity of the first contact surface 113) is made of a material which is sufficiently hard and can prevent bending to an extent that the base body 110 is not deformed even when a large force is applied from the second engaging portion 125 and the second contact surface 122 (described later) of the casing 120. That is, the base body 110 (particularly in the vicinity of the first engaging portion 115 and in the vicinity of the first contact surface 113) has sufficient rigidity which allows the base body 110 to be considered as a rigid body.

### (4) Casing 120

The casing 120 has: the casing body 121 where the second contact surface 122 is formed at a position which corresponds to the first contact surface 113; the second engaging portion 125 formed at a position of an edge side of the casing 120 with respect to the casing body 121; and a casing intermediate portion 123 positioned between the casing body 121 and the second engaging portion 125 (see FIG. 3A).

An inlet port 152 through which blood flows into the blood pump 100 and an outlet port 154 through which blood flows out from the blood pump 100 are formed on the casing body 121. An inner wall (not shown in the drawing) of the casing body 121 defines a portion of the pump chamber 150 (see together with FIG. 1 and FIG. 2).

In the embodiment 1, the inlet port 152 and the outlet port 154 are formed separately from each other. However, the configuration may be adopted where an inlet port and an outlet port form a common port by designing such that a valve or the like is used outside the pump chamber 150.

The second contact surface 122 is a surface where a force directed in a z direction (down) which is generated in the second engaging portion 125 acts on the first contact surface 113 of the base body 110 as a pressing force by way of the casing intermediate portion 123.

One end (upper side) of the casing intermediate portion 123 is continuously formed with the casing body 121, and the other end (lower side) of the casing intermediate portion 123 is continuously formed with the second engaging portion 125. Symbol 124 indicates an intermediate portion inner wall (see FIG. 3A and FIG. 3B).

The casing 120 has a casing edge portion 190 which is positioned on a side opposite to a side of the casing intermediate portion 123 with respect to the second engaging portion 125 (see FIG. 3A and FIG. 3C).

The casing 120 opens on a side where the casing 120 is fitted on the base body 110. The casing edge portion 190, in general, means a portion of an edge which forms the opening.

### (5) Casing Edge Portion 190 and Base Body Stepped Portion 180

As shown in FIG. 3C, on the casing edge portion 190, a casing edge portion lower surface 191 which is a surface of an edge of the casing 120 on a z direction side, a casing edge portion outer surface 192 which is a surface of the edge of the casing 120 on an r direction side, and a casing outer end 193 which is a corner formed by the casing edge portion lower surface 191 and the casing edge portion outer surface 192 are formed.

The casing edge portion lower surface 191 is typically formed in a circular annular shape (doughnut shape) which makes one turn about a rotary axis AX1 of the blood supply mechanism 130 with a predetermined width as viewed along the −z direction.

The casing edge portion outer surface 192 appears on an outer side of the blood pump 100, and forms a surface of the blood pump 100.

As viewed in cross section shown in FIG. 3C, the casing outer end 193 is a corner where the casing edge portion lower surface 191 and the casing edge portion outer surface 192 intersect with each other. Further, in this embodiment, the casing outer end 193 also forms an outer peripheral edge of the casing edge portion lower surface 191, and also forms an end of the casing edge portion 190 on a most r direction side (outer side). However, the casing outer end 193 is not necessarily an end of the whole casing 120 on a most r direction side (outer side).

On the other hand, on the base body stepped portion 180, a base body stepped portion upper surface 181 which is formed in an abutting manner with the casing edge portion lower surface 191 in a state where the casing 120 is fitted on the base body 110, a base body stepped portion outer surface 182 which is a surface of the base body stepped portion 180 on an r direction side, and a base body outer end 183 which is a corner formed by the base body stepped portion upper surface 181 and the base body stepped portion outer surface 182 are formed.

When the casing 120 is fitted on the base body 110, the base body stepped portion upper surface 181 and the casing edge portion lower surface 191 are made to abut against each other (in other words, the base body stepped portion upper surface 181 and the casing edge portion lower surface 191 opposedly facing each other).

In such a state, the base body stepped portion upper surface 181 and the casing edge portion lower surface 191 may be brought into slight contact with each other to an extent that a pressure is not applied to both surfaces. However, at a stage after the casing 120 is fitted on the base body 110 and before welding is performed, it is preferable that the casing edge portion lower surface 191 be not brought into contact with the base body stepped portion upper surface 181 in a direction along the z direction. That is, it is preferable that a slight gap G be formed between the base body stepped portion upper surface 181 and the casing edge portion lower surface 191 (see FIG. 3C). Further, it is preferable that a relationship of G»δ1 be established between the gap G and a gap δ1 (not shown in the drawing) between the contact surfaces (the first contact surface 113 and the second contact surface 122).

An elongation margin (an elongation margin in the z direction) of the casing edge portion 190 referred to as the gap G is ensured in the vicinity of a distal end of the casing edge portion 190. Accordingly, even if the casing is extended in the z direction from the initial state due to a change in temperature or the like, there is no possibility that the casing edge portion lower surface 191 butts against the base body stepped portion upper surface 181 so that the extension of the casing is restricted whereby a pressing force applied to the contact surfaces is decreased.

The base body stepped portion upper surface 181 is typically formed in a circular annular shape (doughnut shape) which makes one turn about the rotary axis AX1 of the blood supply mechanism 130 with a predetermined width as viewed along the z direction.

The base body stepped portion outer surface 182 appears on the outer side of the blood pump 100, and forms the surface of the blood pump 100.

As viewed in cross section shown in FIG. 3C, the base body outer end 183 is a corner where the base body stepped portion upper surface 181 and the base body stepped portion outer surface 182 intersect with each other. Further, in this embodiment, the base body outer end 183 also forms an outer peripheral edge of the base body stepped portion upper surface 181, and also forms a most r direction side (outer side) of the base body stepped portion 180. However, the base body outer end 183 is not necessarily a most r direction side (outer side) of the whole base body 110.

To consider that the blood pump 100 is embedded into the body of a person, it is preferable to adopt the configuration where the casing edge portion outer surface 192 and the base body stepped portion outer surface 182 substantially form the same plane (the casing edge portion outer surface 192 and the base body stepped portion outer surface 182 being brought into a so-called coplanar state). In other words, it is preferable that substantially no step be formed between the casing edge portion outer surface 192 and the base body stepped portion outer surface 182.

### (6) Engagement Between First Engaging Portion 115 and Second Engaging Portion 125

In the blood pump 100 according to the embodiment 1, the casing 120 is fitted on the base body 110 in a slidable manner in the z direction. As a result, the second engaging portion 125 of the casing 120 engages with the first engaging portion 115 of the base body 110 (see FIG. 3B and FIG. 3C).

### (7) Partial Welding

### (7-1) First Profile 183a and Second Profile 193a

Next, the detail of a portion where welding is performed for forming a welded mark 170 is described.

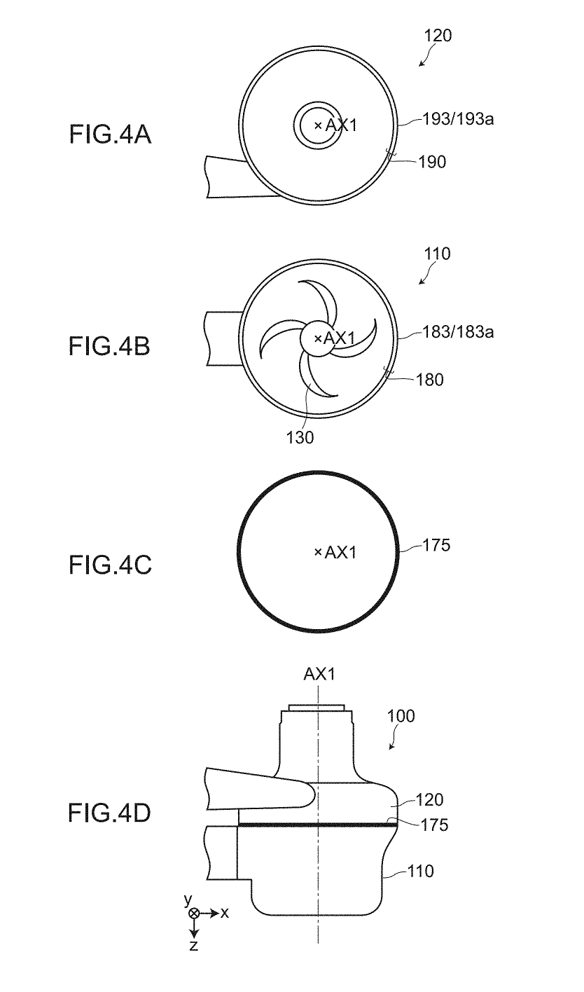

FIG. 4A to FIG. 4D are views for describing an abutting profile 175 which is a candidate for a portion where welding is performed in the embodiment 1. FIG. 4A is a plan view of only the casing 120 as viewed along the z direction, and FIG. 4B is a plan view of only the base body 110 as viewed along the z direction. FIG. 4C is a schematic view expressing a profile (abutting profile 175) which is a logical product of the first profile 183a and the second profile 193a described later when the blood pump 100 is viewed along the z direction in a state where the casing 120 is fitted on the base body 110. FIG. 4D is a side view of the blood pump 100 as viewed along the y direction.

In this embodiment, a profile of the base body outer end 183 when the base body 110 is viewed along the z direction is defined as “first profile 183a”. As shown in FIG. 4B, the first profile 183a is formed so as to surround the rotary axis AX1 of the blood supply mechanism 130.

A profile of the casing outer end 193 when the casing 120 is viewed along the z direction is defined as “second profile 193a”. As shown in FIG. 4A, the second profile 193a is formed so as to surround the rotary axis AX1 of the blood supply mechanism 130.

In the above-mentioned “so as to surround” includes not only a case where the profile is continuously formed thus forming a circular shape as shown in FIG. 4A and FIG. 4B but also a case where a portion of the profile is interrupted so that the profile is formed discontinuously although the profile surrounds the rotary axis AX1 as a whole (see modification 4 described later).

### (7-2) Abutting Profile 175

When the blood pump 100 is viewed along the z direction in a state where the casing 120 is fitted on the base body 110, a profile formed by a logical product of the first profile 183a of the base body outer end 183 and the second profile 193a of the casing outer end 193 is defined as “abutting profile 175” (see FIG. 4C).

The abutting profile 175 is a profile which is a sum of a group of candidates of portions where welding (described later) is performed. In performing welding, welding is performed by selecting arbitrary portions of the abutting profile 175. However, the candidates for the portions where welding is performed are not always limited to portions within the abutting profile 175.

A specific example of the abutting profile 175 is described. Assume a case where the base body stepped portion upper surface 181 and the casing edge portion lower surface 191 respectively form a circular annular shape (doughnut shape) which forms one turn about the rotary axis AX1 of the blood supply mechanism 130 with a predetermined width. When a state where the base body stepped portion upper surface 181 and the casing edge portion lower surface 191 abut against each other is viewed along the z direction, the abutting profile 175 corresponds to a logical product of an outer periphery of a circular ring of the base body stepped portion upper surface 181 and an outer periphery of a circular ring of the casing edge portion lower surface 191. In a case where the outer peripheries of the base body stepped portion upper surface 181 and the casing edge portion lower surface 191 are continuously formed and form a circular shape, a result of the logical product (abutting profile 175) forms a circular shape which forms a continuous one turn about the rotary axis AX1 (see FIG. 4C and FIG. 4D).

For reference, in a case that a substantially same plane is formed by the casing edge portion outer surface 192 and the base body stepped portion outer surface 182, when the first profile 183a and the second profile 193a are viewed along the plane, the first profile 183a and the second profile 193a appear in an overlapping manner, and form the abutting profile 175.

### (7-3) Welded Mark 170 Connecting First Profile 183a and Second Profile 193a

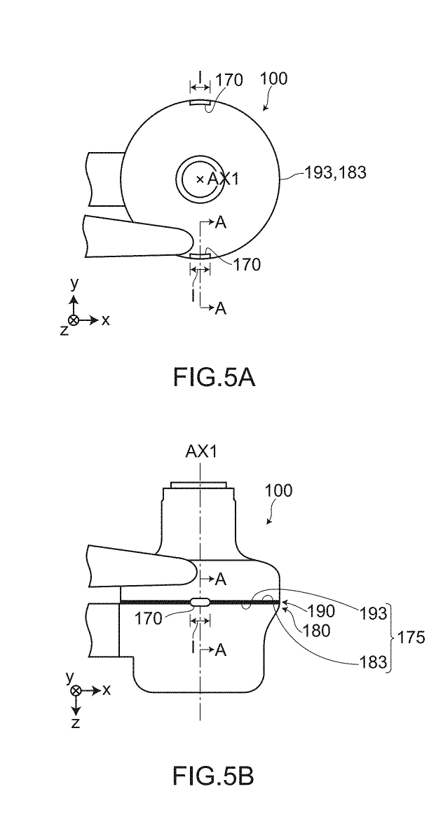

FIG. 5A and FIG. 5B are views for describing portions where a welded mark is disposed and a length l (“l” being lower case L) of the welded mark 170 in the blood pump 100 according to the embodiment 1. FIG. 5A is a plan view of the blood pump 100 as viewed along the z direction, and FIG. 5B is a side view of the blood pump 100 as viewed along the y direction.

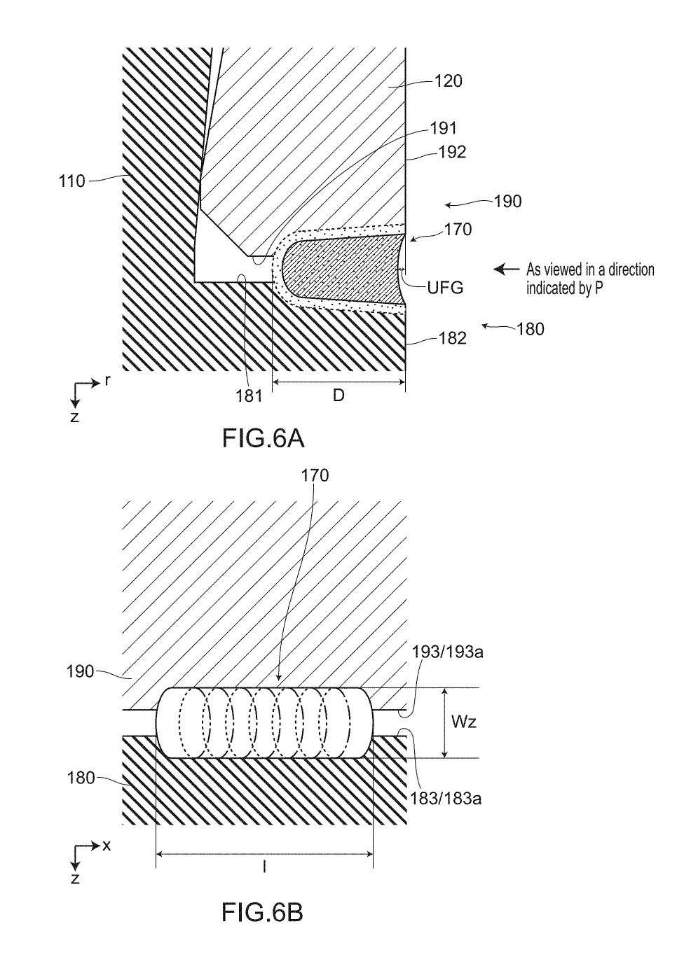

FIG. 6A and FIG. 6B are views for describing the welded mark 170 in the embodiment 1. FIG. 6A is a cross-sectional view taken along a line A-A in FIG. 5. FIG. 6B is a view where the welded mark 170 and an area around the welded mark 170 are viewed from a P side in FIG. 6A.

As a result of welding where welding is applied to predetermined positions within the above-mentioned abutting profile 175, the blood pump 100 has the welded marks 170 which connect the casing edge portion 190 and the base body stepped portion 180 to each other between the first profile 183a of the base body outer end 183 and the second profile 193a of the casing outer end 193 at least at two portions which are separated from each other (see FIG. 5A to FIG. 6B).

In other words, the blood pump 100 has the welded mark 170 which connects the casing edge portion 190 and the base body stepped portion 180 at least at two portions on the circumference where the abutting profile 175 exists (the portions where the above-mentioned logical product becomes true).

Further, the blood pump 100 has the plurality of these welded marks 170 at the positions separated from each other. In other words, the plurality of these welded marks 170 are arranged at positions separated from each other. That is, in the blood pump 100 according to the embodiment 1, welding is not performed over the whole circumference of the abutting profile 175 formed of the first profile 183a and the second profile 193a (whole circumference welding), and welding is performed on portions of the circumference in a state that portions where welding is not performed remain (partial welding).

### (7-4) Structure of Welded Mark 170

When viewed macroscopically, the welded mark 170 connects casing edge portion 190 and the base body stepped portion 180 (see FIG. 5A and FIG. 5B).

When viewed microscopically, metal materials which form the first profile 183a and the second profile 193a are melted and fused together thus forming the welded mark 170, and the welded mark 170 connects the casing edge portion 190 and the base body stepped portion 180 such that the welded mark 170 spans between the casing edge portion 190 and the base body stepped portion 180 (see FIG. 6A and FIG. 6B).

The welded mark 170 is formed by laser welding or the like, for example, by using the casing 120 (to be more specific, the casing edge portion 190, for example) and the base body 110 (to be more specific, the base body stepped portion 180, for example) as base materials.

A main part of the welded mark 170 is formed of a portion (weld bead) where a portion of the casing edge portion 190 and a portion of the base body stepped portion 180 are melted and, thereafter, are solidified. Around the portion where the base materials are melted and, thereafter, are solidified, a portion may be formed where a portion of the casing edge portion 190 and a portion of the base body stepped portion 180 are changed in property by a thermal effect (not indicated by symbols in the drawing).

The welded mark 170 penetrates in the −r direction with a depth D (see FIG. 6A).

The welded mark 170 has a width Wz in the z direction (substantially equal to a so-called surface bead width). Further, the welded mark 170 has a length l per one place (see FIG. 6B). A total length of the welded marks 170 becomes L over the whole blood pump 100.

“total length L of the welded marks 170” is a sum of lengths l (“l” being lower case L) each of which is a length of the welded mark 170 per l place. For example, in the case where the casing edge portion 190 and the base body stepped portion 180 are formed in a circular shape as viewed along the z direction, “a total length L of the welded marks 170” becomes a total extension length of the welded marks 170 in the circumferential direction about the rotary axis AX1.

### (7-5) Size Relationship of Welded Mark 170

A surface of the welded mark 170 on an r direction side may be continuously formed between the casing edge portion outer surface 192 and the base body stepped portion outer surface 182 (that is, an underfill being eliminated substantially).

An underfill may be formed on the welded mark 170. In this case, it is preferable that a gap UFG (an underfill gap UFG) between a deepest portion of the underfill and the casing edge portion outer surface 192 or the base body stepped portion outer surface 182 fall within a range of from 0.05 mm to 0.3 mm (see FIG. 6A).

The underfill gap UFG takes a value in a positive range and hence, a weld bead which forms the welded mark 170 does not protrude from the casing edge portion outer surface 192 or the base body stepped portion outer surface 182 on an r direction side (outer side) whereby there is no possibility that the welded mark 170 obstructs embedding of the blood pump into the body of a user.

Further, the underfill gap UFG is 0.3 mm or less and hence, a welding depth can be ensured compared to a case where the underfill gap FUG exceeds 0.3 mm and hence, a casing peel-off strength can be further enhanced.

It is preferable that a width Wz of the welded mark 170 in the z direction fall within a range of from 0.3 mm to 2.0 mm, and

a total length of the welded marks 170 fall within a range of from 2 mm to 40 mm when the welded marks 170 are viewed along the z direction (see FIG. 6B).

By connecting the casing edge portion and the base body stepped portion by welding such that the welded mark 170 is formed at least two portions or more, a required casing peel-off strength can be obtained first. Then, by setting a width of the welded mark 170 to a value which falls within a range of from 0.3 mm to 2.0 mm and by setting a total length of the welded marks 170 to a value which falls within a range of from 2 mm to 40 mm, a casing peel-off strength can be further enhanced.

It is preferable that the total length of the welded marks 170 be 5 mm or more when the welded marks 170 are viewed along the z direction.

By setting a total length of the welded marks to 5 mm or more, the casing and the base body can be joined more strongly thus further enhancing a casing peel-off strength.

It is preferable that the total length of the welded marks 170 be 30 mm or less when the welded marks 170 are viewed along the z direction.

A total length of the welded marks 170 is 30 mm or less (an average length per one portion of the welded mark 170 being 15 mm or less when the welded mark 170 is formed at two portions). Accordingly, for example, in a step of disassembling the blood pump 100, a length of removing by cutting (fusing, cutting or the like) at the time of separating a portion of the casing 120 or/and the base body 110 including the welded mark 170 from other portions which do not include the welded mark 170 becomes 15 mm or less in average and hence, the separating operation can be performed relatively easily.

Further, even if the blood pump is a miniaturized blood pump where a length of the circumference of the abutting profile 175 formed by the first profile 183a and the second profile 193a is approximately 100 mm (a diameter of the casing when viewed along the z direction being approximately 35 mm), provided that a total length of the welded marks 170 is approximately 30 mm or less which is approximately ⅓ or less of the length of the circumference of the abutting profile 175, welding can be performed at least twice. That is, by setting a total length of the welded marks 170 to 30 mm or less, a series of steps consisting of disassembling of the blood pump (blood pump disassembling step), analysis of the inside of the blood pump, working, readjustment and the like (blood pump analyzing step), and welding at portions different from the portions where welding is performed previously (rewelding step) can be performed at least one cycle.

Due to the same reason set forth above, it is preferable that a total length of the welded marks 170 be set to a value which falls within a range of from 1% to 40% with respect to the length of the periphery (becoming candidates for portions to which welding is applied) of the abutting profile 175 formed of the first profile 183a and the second profile 193a. It is more preferable that a total length of the welded marks 170 be set to 3% or more with respect to the length of the above-mentioned periphery. It is still more preferable that a total extension length of the welded marks 170 be set to 20% or less with respect to the length of the above-mentioned periphery.

(7-6) The Number of Portions where Welded Mark 170 is Arranged and the Arrangement of Welded Marks 170

In the embodiment 1, the number of portions where the welded mark 170 is arranged is two or more.

However, as shown in FIG. 5A, the number of portions where the welded mark 170 is arranged is two. When the number of portions where the welded mark 170 is arranged is two, the number of such portions can be suppressed to minimum while maintaining a required casing peel-off strength and hence, disassembling (blood pump disassembling step) of the blood pump can be performed relatively easily. Further, by setting the number of portions where welded mark is arranged to a minimum value of two, even in performing the rewelding step, the number of selections of the portions where rewelding is performed (portions different from the portions to which welding is applied previously) is large and hence, rewelding step can be easily performed.

When the number of portions where the welded mark 170 is arranged is two, as shown in FIG. 5A, the welded marks 170 may be arranged in point symmetry with respect to the rotary axis AX1. When the number of portions where the welded mark 170 is arranged is n (n being an integer of 2 or more), the welded marks 170 may be arranged on the abutting profile 175 at an equal angular pitch of 360°/n about the rotary axis AX1.

The welded marks 170 may be arranged by other methods instead of the equal angular pitch arrangement. To be more specific, the welded marks 170 may be arranged at non-equal angular pitches depending on shapes of the base body stepped portion 180 and the casing edge portion 190 as viewed along the z direction, the deviation of a thickness of the casing edge portion 190 in the r direction or the like.

### (8) Pressing of Second Contact Surface to First Contact Surface

In the blood pump 100 according to the embodiment 1, the second contact surface 122 of the casing 120 is pressed so as to be brought into contact with the first contact surface 113 of the base body 110.

In this embodiment, “pressed” does not mean a state where the second contact surface 122 simply butts against the first contact surface 113 but means a state where a pressing force is constantly applied to the second contact surface 122 and the first contact surface 113.

The first contact surface 113 and the second contact surface 122 are strongly pressed to each other by a pressing force while being brought into contact with each other and hence, it is possible to prevent blood in the pump chamber 150 from intruding between the first contact surface 113 and the second contact surface 122.

In this embodiment, “butting” is also referred to as “wholly brought into contact with each other” or the like. In a state where, for example, when a force is applied to a unit in the z direction, the unit and a counter unit which opposedly faces the unit are brought into contact with each other at a portion (or at a surface or the like), and a force in the z direction is applied to the whole surface of the counter unit at the portion (at the surface), such a portion (surface) is referred to as a “butting” portion (surface).

### 2. Detailed Structure of Other Parts of Blood Pump 100

Hereinafter, the description is made with respect to the detailed structure relating to the fitting engagement between the casing 120 having a tapered shape or the like and the base body 110.

FIG. 7 is a view for describing a manner in which a pressing force F2 which acts from a second contact surface 122 to a first contact surface 113 is generated in the blood pump 100 according to the embodiment 1.

FIG. 8 is a view for describing a size relationship of the main part of the blood pump 100 according to the embodiment 1.
(1) In the blood pump 100 according to the embodiment 1, a first tapered portion 116 inclined toward the −r direction (inner side) as the first tapered portion 116 extends from a tapered outer end portion 117 in the z direction (down) is formed on the first engaging portion 115. A second tapered portion 126 inclined toward the r direction (outer side) as the second tapered portion 126 extends from a tapered inner end portion 127 in the −z direction (up) on an inner wall (not shown in the drawing) of the casing 120 is formed on the second engaging portion 125 (see FIG. 3A and FIG. 3B).

In the first tapered portion 116, the tapered outer end portion 117 protrudes in the most r direction side (outer side) of the first tapered portion 116, and the first tapered terminal end portion 118 is disposed at a position on a most −r direction side (inner side) of the first tapered portion 116. In the second tapered portion 126, the tapered inner end portion 127 protrudes toward the most −r direction side (inner side) of the second tapered portion 126, and the second tapered terminal end portion 128 is disposed at a position on the most r direction side (outer side) of the second tapered portion 126.

In the blood pump 100 according to the embodiment 1, the first engaging portion 115 having the first tapered portion 116 and the second engaging portion 125 having the second tapered portion 126 engage with each other (see 3B, FIG. 3C, and FIG. 7).

Before such a state is brought about, in a casing fitting step S30 of the blood pump 100 (described later), the casing 120 is fitted on the base body 110 by press fitting from an extremely tight state.

When the casing 120 is fitted on the base body 110, as shown in FIG. 7, an edge side of the casing 120 is elastically deformed by being expanded in the r direction side (outer side) as a whole compared to a state before fitting. Due to such elastic deformation, an elastic force F1 (a force which fastens the base body 110) which intends to return in the −r direction (inner side) is generated. Particularly, an extremely large elastic force F1 acts on the first tapered portion 116 around an area in the vicinity of the tapered inner end portion 127 of the second engaging portion 125. On the other hand, a base body 110 side including the first tapered portion 116 is regarded as a rigid body as described above. Accordingly, when the elastic force F1 acts, reversely, a resistance force perpendicular to the second engaging portion 125 is generated in a direction perpendicular to a contact surface of the first tapered portion 116. Along with the generation of the resistance force, due to an effect of the inclined surface by the first tapered portion 116, a force F2 which is a component in the z direction (downward direction) of the perpendicular resistance force is also generated. This force F2 is a force which presses down the whole casing 120 in the z direction (down). In an interlocking manner with such a pressing operation, a large pressing force F2 is applied from the second contact surface 122 on a casing 120 side to the first contact surface 113 on a base body 110 side.
(2) In the blood pump 100 according to the embodiment 1, assuming an inner diameter of the tapered inner end portion 127 of the second tapered portion 126 of the casing 120 as ϕA and an outer diameter of the tapered outer end portion 117 of the first tapered portion 116 of the base body 110 as ϕB before the casing 120 is fitted on the base body 110, a relationship of ϕA<ϕB is established. The casing 120 is fitted on the base body 110 in a state that a position of the tapered inner end portion 127 of the second tapered portion 126 is disposed at a portion of the first tapered portion 116 shifted in a z direction side from the tapered outer end portion 117, the portion being a portion of the first tapered portion 116 away a first tapered terminal end portion 118 on a side opposite to the tapered outer end portion 117(see FIG. 8 and FIG. 3B).

That is, the tapered inner end portion 127 of the casing 120 is in a state where the tapered inner end portion 127 is disposed at a position on the z direction (down) side getting over the tapered outer end portion 117 of the base body 110.

The description of a mode in which the casing 120 is fitted on the base body 110 is broken up into groups of sentences. First, the casing 120 is made to slide in the z direction with respect to the base body 110, and at a moment when the tapered inner end portion 127 of the casing 120 gets over the tapered outer end portion 117 of the base body 110, an edged side of the casing 120 is expanded and is deformed at maximum in the r direction (outer side) thus generating a large resilient force.

Further, even when sliding of the casing 120 advances and the tapered inner end portion 127 reaches a portion of the base body 110 where the tapered inner end portion 127 is shifted in the z direction side from the tapered outer end portion 117, an elastic force is maintained. At this state of operation, due to an effect of the first tapered portion 116 described above, the elastic force is converted into a force which presses the second engaging portion 125(and/or the whole casing) of the casing 120 in a z direction (down) by way of the tapered inner end portion 127.

The pressing force which presses in the z direction can be further added as a portion of the pressing force on the contact surface and, at the same time, contributes to the enhancement of a casing peel-off strength.

The position of the tapered inner end portion 127 is disposed at the position shifted from the tapered outer end portion 117 in the z direction side and hence, it is difficult for the tapered inner end portion of the casing 120 to move in the −z direction (up). Accordingly, it is always possible to constantly add a force to the above-mentioned pressing force in a stable manner.

By adopting the above-mentioned configuration, it is possible to stably obtain a pressing force and a casing peel-off strength in the blood pump 100.
(3) In the blood pump 100 according to the embodiment 1, it is preferable that, assuming an angle made by a profile of an inclined surface of the first tapered portion 116 and the z direction as ϕ1 and an angle made by a profile of an inclined surface of the second tapered portion 126 and the z direction as ϕ2, a relationship of ϕ1≤ϕ2 be established (see FIG. 8, FIG. 3B, and FIG. 3C).

In the case where the relationship of ϕ1<ϕ2 is established, the tapered inner end portion 127 of the casing 120 is brought into contact with the first tapered portion 116 of the base body 110. With such a configuration, in fitting the casing 120 on the base body 110 by sliding the casing 120 in the z direction, an elastic force generated due to the deformation of the casing 120 (and the above-mentioned force which presses the casing 120 to the base body 110 in the z direction generated by the elastic force) is concentrated on a portion of the tapered inner end portion 127 which is brought into contact with the first tapered portion 116.

Accordingly, by properly setting the relative position of a casing inner end portion of the casing, the above-mentioned force for pressing the casing 120 to the base body 110 in the z direction can be controlled in a stable manner and with high precision.

By adopting such a configuration, it is possible to obtain a pressing force and a peel-off strength in a stable manner and with high precision.

Even when the angle ϕ1 and the angle ϕ2 are substantially equal, an elastic force is generated by a tapering effect and an additional force is applied to a pressing force.

Further, in the blood pump 100 according to the embodiment 1, it is preferable that the relationship of 0°≤ϕ1 be satisfied and the relationship of ϕ2≤20° be satisfied.

When the angle ϕ1 is set to 0° (ϕ1=0°, fitting of the casing 120 on the base body 110 and the removal of the casing 120 from the base body 110 becomes relatively easy.

Further, it is preferable that a difference between the angles ϕ1 and ϕ2 satisfy the relationship of 2°≤(ϕ2−ϕ1)≤10°.
(4) In the blood pump 100 according to the embodiment 1, it is preferable that assuming a thickness of the casing body 121 in a direction perpendicular to a tangent plane on an outer side of the casing body 121 as t1, a thickness of the casing intermediate portion 123 in a direction perpendicular to a tangent plane of an outer side of the casing intermediate portion 123 as t2, and a thickness of the second engaging portion 125 in a direction perpendicular to a tangent plane of an outer side of the second engaging portion 125 as t3, a relationship of t1>t2>t3 be established. Further, it is preferable that, with respect to an inclined surface which forms the second tapered portion 126 of the second engaging portion 125, on a second tapered terminal end portion 128 side which is a side opposite to the tapered inner end portion 127, the casing have a smallest thickness (see FIG. 8 and FIG. 3A).

With such a configuration, the thickness of the casing 120 is gradually decreased in a stepwise manner from the casing body 121 to the second engaging portion 125 by way of the casing intermediate portion 123. Accordingly, although a deformation amount of the casing intermediate portion 123 is smaller than a deformation amount of the second engaging portion 125, the casing intermediate portion 123 is elastically deformed to some extent so that it is possible to efficiently impart a strong elastic force to the casing 120 as a whole similar to a fishing rod.

In the blood pump 100 according to the embodiment 1, it is preferable that assuming a thickness of the casing intermediate portion 123 in a direction perpendicular to the tangent plane of the outer side of the casing intermediate portion 123 which is a thickness of a portion of the casing 120 having a smallest thickness as t2′, and assuming a thickness of the second engaging portion 125 in a direction perpendicular to the tangent plane of the outer side of the second engaging portion 125 as t3, a relationship of t2′<t3 be established. In other words, it is preferable that the casing intermediate portion 123 have a smallest thickness at a middle portion thereof thus forming a neck portion 129 which become a “neck”, and a thickness t2′ of the neck portion 129 be set smaller than a thickness t3 of the second engaging portion 125 (see FIG. 8, FIG. 3A, and FIG. 3B).

Due to the formation of the neck portion 129 having such a thickness relationship, an edge side of the casing formed of the casing intermediate portion 123, the second engaging portion 125, and the casing edge portion 190 can be easily deformed and hence, the casing 120 can be easily fitted on the base body 110.

Further, due to the formation of such a neck portion 129, the casing 120 can exhibit an elastic force even after the casing 120 is fitted on the base body 110. Accordingly, joining between the first engaging portion 115 and the second engaging portion 125 by engagement becomes strong thus further enhancing a casing peel-off strength.

Further, the neck portion 129 has a smaller thickness than other portions. Accordingly, in separating a portion of the casing which includes the welded marks 170 from other portions which do not include the welded marks 170 for disassembling the blood pump 100 (at the time of performing the welded mark separation step), it is sufficient to remove by cutting (fusing, cutting or the like) the casing at a portion of the neck portion 129. Accordingly, such a separation operation can be performed extremely easily.
(5) In the blood pump 100 according to the embodiment 1, in a state where the first engaging portion 115 and the second engaging portion 125 are cut along an xy plane and are viewed in a plan view along the z direction, the first engaging portion 115 and the second engaging portion 125 are formed in a circular shape respectively. In this embodiment, “circular shape” means that the shape has no corners, and an elliptical shape, a true circular shape and the like are named as “circular shape”.

In this manner, by forming the first engaging portion 115 and the second engaging portion 125 in a circular shape, when the casing 120 is fitted on the base body 110, an elastic force is uniformly generated as a whole so that a pressing force is also uniformly generated whereby it is possible to provide the blood pump 100 having the stable fitting engaging structure.

In the blood pump 100 according to the embodiment 1, the second engaging portion 125 of the casing 120 may have the structure where a “slit” is formed along an outer periphery of the second engaging portion 125. However, it is preferable that the second engaging portion 125 have the continuous ring-shaped structure (approximately circular cylindrical shape) having no “slit”.

With such a structure, when the tapered inner end portion 127 of the casing 120 gets over the tapered outer end portion 117 of the base body 110, the second engaging portion 125 can generate a larger elastic force than the second engaging portion having “slit”. With such a structure, even when thicknesses of the second engaging portion 125, the casing intermediate portion 123 and the like of the casing 120 are set small (thin), the casing 120 can ensure a sufficient elastic force. Such a structure also contributes to the miniaturization and the reduction of weight of the blood pump 100.
(6) In the blood pump 100 according to the embodiment 1, a mirror finish is applied to the approximately horizontal surface 111 of the base body 110. For example, it is preferable that a value of Ra (arithmetic average roughness) be approximately 1.0 or less, and it is more preferable that a value of Ra (arithmetic average roughness) be approximately 0.2 or less. With such a configuration, the first contact surface 113 which forms a part of the approximately horizontal surface 111 is brought into contact with the second contact surface 122 with a smoother surface and hence, it is possible to make the formation of a gap between the first contact surface 113 and the second contact surface 122 more difficult. Further, a mirror finish is also applied to the blood contact surface 112 which forms a part of the approximately horizontal surface 111 of the base body 110 and hence, blood minimally adheres to the surface whereby the generation of a thrombus by a congest can be further effectively prevented.

“approximately horizontal surface 111” means a surface which becomes horizontal when the blood pump 100 is placed in a usual state. However, “approximately horizontal surface 111” also includes a case where “horizontal” has slight deviation. Further, even when “approximately horizontal surface 111” is not horizontal (vertical or the like) in view of designing or a use state, in this embodiment, such a state is also defined as “horizontal surface” or “approximately horizontal surface” for the sake of convenience. Further, “approximately horizontal surface 111” also includes a case where the plane 111 is not formed of a completely flat surface. For example, the blood contact surface 112 may be plane which draws a slight curve.
(7) The casing 120 may be formed using any material which can be used for forming the blood pump 100 according to the embodiment 1. For example, it is possible to adopt materials such as pure titanium (F67, grade 2 or the like stipulated in ASTM standard, second type stipulated in JIS standard or the like), a titanium alloy, stainless steel (SUS or the like), or other various alloys. Further, in forming the second engaging portion, it is possible to use a material belonging to a resin provided that the second engaging portion is made of such a material which can generate an elastic force thus generating a required pressing force.

However, in the embodiment 1, it is preferable that the casing 120 be made of a material which contains titanium as a main component. Titanium is a material whose biocompatibility is confirmed and is permitted as a medical-use material which can be incorporated into a human body. Titanium has a sufficient tensile strength, a sufficient yield strength, and a high specific strength. Accordingly, even when a thickness of the casing made of titanium is made small, the casing exhibits a high strength and is light-weighted, and has appropriate elasticity. Titanium has physical properties suitable for forming the casing according to the present invention. From various viewpoints including the above-mentioned viewpoints, with the use of titanium as a material for forming the casing, it is possible to provide a blood pump for a highly rational auxiliary artificial heart.

### 3. Method of Manufacturing Blood Pump 100/100a According to Embodiment 1

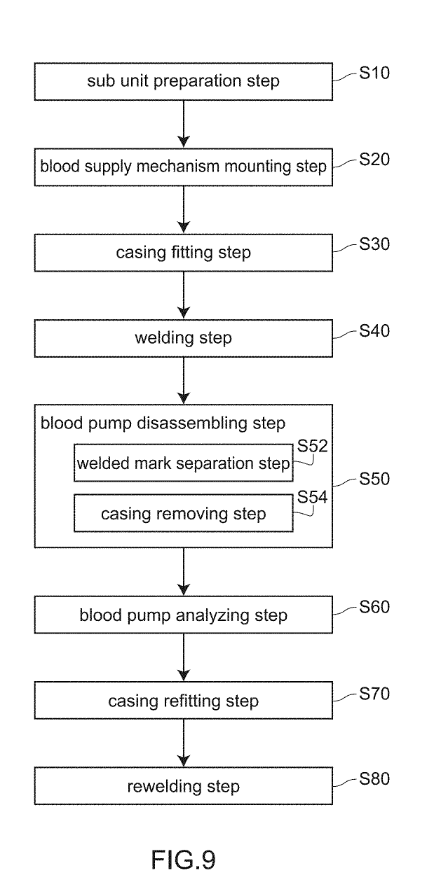

FIG. 9 is a flowchart for describing a method of manufacturing a blood pump according to the embodiment 1.

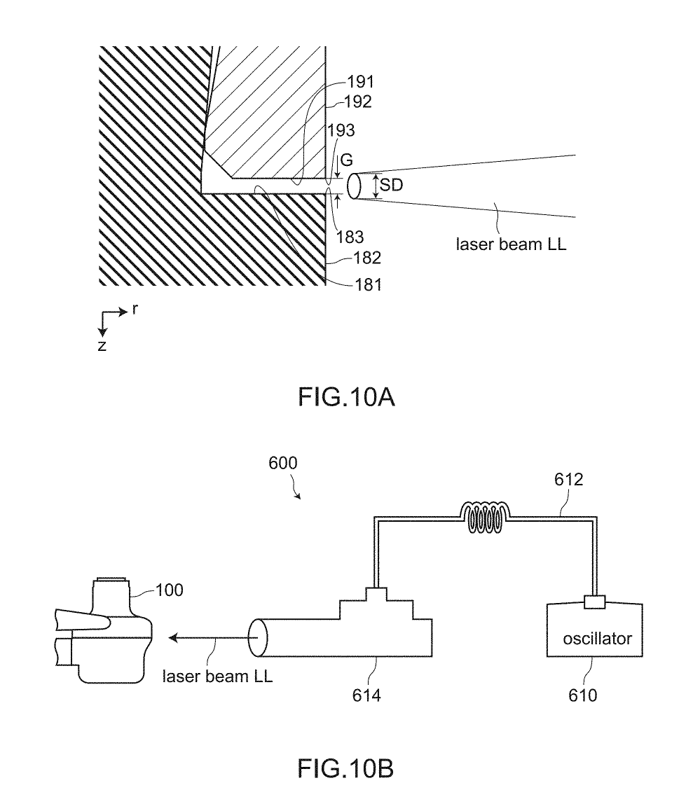

FIG. 10A and FIG. 10B are views for describing a welding step S40 in the embodiment 1.

In the method of manufacturing the blood pump 100 according to the embodiment 1, as shown in FIG. 9, the method includes a sub unit preparation step S10, a blood supply mechanism mounting step S20, the casing fitting step S30, and the welding step S40 in this order. Hereinafter, the respective steps are described in this order.

### (1) Sub Unit Preparation Step S10

Firstly, a sub unit group which forms the blood pump 100 is prepared (sub unit preparation step S10). To be more specific, the base body 110, the casing 120, and the blood supply mechanism 130 which form separate parts from each other are prepared.

### (2) Blood Supply Mechanism Mounting Step S20

Next, the blood supply mechanism 130 is mounted on the base body 110 (blood supply mechanism mounting step S20). With such an operation, for example, as shown in FIG. 1, a state is brought about where the blood supply mechanism 130 is integrally mounted on the base body 110.

### (3) Casing Fitting Step S30

Next, the casing 120 is fitted on the base body 110 such that the second engaging portion 125 engages with the first engaging portion 115 by sliding the casing 120 in the z direction with respect to the base body 110 (casing fitting step S30).

To be more specific, the second engaging portion 125, the casing edge portion 190 and the like of the casing 120 are made to slide in the z direction (from the upper direction to the lower direction) such that the second engaging portion 125, the casing edge portion 190 and the like are moved toward and are fitted on the first engaging portion 115, base body stepped portion 180 and the like of the base body 110. With such an operation, a state is brought about where the second engaging portion 125 engages with the first engaging portion 115.

When the second engaging portion 125 and the first engaging portion 115 are formed into a tapered shape as shown in FIG. 3A to FIG. 3C, for example, the tapered inner end portion 127 of the casing 120 is made to get over the tapered outer end portion 117 of the base body 110 and is made to further slide to a position in the z direction (down). With such an operation, the casing 120 is fitted on the base body 110 in a state where the tapered inner end portion 127 of the second tapered portion 126 is positioned at a portion of the first tapered portion 116 shifted to a z direction side from the tapered outer end portion 117 and away the first tapered terminal end portion 118 on a side opposite to the tapered outer end portion 117 of the first tapered portion 116.

On the other hand, for example, in a case where the second engaging portion 125 and the first engaging portion 115 are formed into a non-tapered shape (referred to as “straight shape” for the convenience's sake) in an embodiment 2 described later, it is assumed that the casing 120 is fitted on the base body 110 although the fitting engagement receives a slight resistance force.

### (4) Welding Step S40

Next, welding is performed so as to connect the casing edge portion 190 and the base body stepped portion 180 to each other at least at two portions which are separated from each other while pressing the casing 120 to the base body 110 in the z direction (welding step S40).

### (4-1) Abutting Between Casing Edge Portion Lower Surface 191 and Base Body Stepped Portion Upper Surface 181

Prior to welding, the above-mentioned casing fitting step S30 is performed so as to bring about a state where the casing edge portion 190 approaches the base body stepped portion 180.

For example, the casing edge portion lower surface 191 and the base body stepped portion upper surface 181 are made to opposedly face each other in an abutting manner and hence, a state is brought about where the casing edge portion 190 approaches the base body stepped portion 180 (estimating abutting welding).

The casing edge portion lower surface 191 may be brought into contact with the base body stepped portion upper surface 181.

However, to take into account the following (1) to (4), it is preferable to form a gap G between the casing edge portion lower surface 191 and the base body stepped portion upper surface 181 (see FIG. 10A).

(1) Ensuring an elongation margin which allows the casing edge portion lower surface 191 to be shifted in the direction (down) toward the base body stepped portion upper surface 181 when the casing 120 is pressed to the base body 110

(2) Ensuring an elongation margin which allows the downward extension of the casing edge portion lower surface 191 when the extension and shrinkage of a material of the casing 120 is estimated

(3) Enhancement of welding efficiency

(4) Irregularities in manufacture of the blood pump 100

However, when the gap G is excessively large, an underfill gap UFG (see FIG. 6A) of the welded mark 170 formed after welding becomes excessively large and hence, a casing peel-off strength is decreased eventually. Accordingly, it is preferable that the gap G be smaller than a predetermined size.

To take into account the above-mentioned point of view, it is preferable that the gap G formed between the casing edge portion lower surface 191 and the base body stepped portion upper surface 181 be larger than 0 mm and equal to or less than 150 μm, and the gap G formed between the casing edge portion lower surface 191 and the base body stepped portion upper surface 181 be set to approximately 5 μm, for example.

### (4-2) Means for Performing Welding

As described above, the casing edge portion 190 and the base body stepped portion 180 are connected to each other by welding in a state where the casing edge portion 190 and the base body stepped portion 180 approach each other or are brought into contact with each other.

As the means for performing welding, a so-called fiber laser 600 may be adopted. In the fiber laser 600, a beam is excited by a semiconductor laser of an oscillator 610, the excited beam is introduced to an amplifier in a fiber 612 so as to amplify the beam, and a laser beam LL is irradiated from an irradiation unit 614 (see FIG. 10B).

In the above-mentioned fiber laser 600, a beam is excited by the semiconductor laser of high efficiency and hence, a welding time becomes short, and heating of the base body 110 and the casing 120 which form a base material can be suppressed to a minimum and hence, a damage by heat to the base body 110, the casing 120, a packing 160 (described later) and the like can be suppressed to a minimum. Further, continuous laser irradiation can be performed by continuous oscillation and hence, a smooth welded mark can be obtained. Still further, key-hole-type welding or deep penetration welding can be performed and hence, it is possible to obtain a welded mark (bead) having a sufficient depth D while minimizing a width Wz of the welded mark (bead).

### (4-3) Position at Which Welding is Performed

For example, in a case where the casing edge portion lower surface 191 and the base body stepped portion upper surface 181 are made to opposedly face each other in a state where the casing edge portion lower surface 191 abuts against the base body stepped portion upper surface 181, welding is performed between the second profile 193a which the casing outer end 193 forms and the first profile 183a which the base body outer end 183 forms.

A position of the center of a spot diameter SD of the laser beam LL is set such that the center of the spot diameter SD of the laser beam LL is positioned in the vicinity of a middle point between the casing outer end 193 and the base body outer end 183 as viewed along the z direction (as viewed along the casing edge portion outer surface 192 or the base body stepped portion outer surface 182) (see FIG. 10A).

In this embodiment, it is preferable to set a relationship between the gap G formed between the casing edge portion lower surface 191 and the base body stepped portion upper surface 181 (or the gap G formed between the casing outer end 193 and the base body outer end 183) and the spot diameter SD of the laser beam LL to G≤1.5×SD. For example, it is preferable that the gap G be 150 μm or less when the spot diameter SD is 100 μm.

It is not always necessary that the position of the center of the spot diameter SD of the laser beam LL in the z direction be disposed in the vicinity of the middle point between the casing outer end 193 and the base body outer end 183. The position of the center of the spot diameter SD of the laser beam LL can be determined by suitably shifting the position of the center of the spot diameter SD of the laser beam LL in the z direction depending on wall thicknesses, materials or the like of the casing edge portion 190 and the base body stepped portion 180.

### (4-4) Irradiation of Laser Beam LL

In a state where the position of the center of the spot diameter SD of the laser beam LL is determined as described above, the laser beam LL is irradiated toward an area in the vicinity of the casing edge portion 190 and the base body stepped portion 180 so as to melt a portion of the casing edge portion 190 which forms a base material and a portion of the base body stepped portion 180 which forms a base material.

In performing welding using the above-mentioned fiber laser 600, welding is performed by a so-called key-hole-type laser welding or deep penetration laser welding. By performing welding using such laser welding, the welded mark 170 which penetrates in the −r direction with a depth D can be obtained and hence, the casing 120 can be strongly welded to the base body 110 (see FIG. 6A).

The position at which the laser beam LL is irradiated is moved such that scanning is performed along the circumferences of the casing outer end 193 and the base body outer end 183 as viewed along the z direction. The position at which the laser beam LL is irradiated may be moved such that the welded mark 170 is formed over a length of l mm (“l” being lower case L) per each portion to which welding is applied (see FIG. 6B).

In this embodiment, the example is described where laser welding is adopted as the means for performing the welding step S40. However, in the embodiment 1, the means for performing welding is not limited to such welding. For example, the welding step S40 can be performed by gas welding, arc welding, resistance welding or the like.

When the fiber laser 600 is used as the means for performing welding, it is preferable that a width Wz of the welded mark 170 in the z direction fall within a range of from approximately 0.3 mm to 1.0 mm. It is more preferable that the width Wz of the welded mark 170 in the z direction be set to 0.5 mm or more. It is further preferable that the width Wz of the welded mark 170 in the z direction be set to 0.8 mm or less. It is still further preferable that the width Wz of the welded mark 170 in the z direction fall within a range of from 0.5 mm to 0.7 mm.

When a CO2 laser (not shown in the drawing) is used as the means for performing welding, it is preferable that a width Wz of the welded mark 170 in the z direction fall within a range of from approximately 0.7 mm to 2.0 mm. It is more preferable that the width Wz of the welded mark 170 in the z direction be set to 1.0 mm or more. It is further preferable that the width Wz of the welded mark 170 in the z direction be set to 1.5 mm or less. It is still further preferable that the width Wz of the welded mark 170 in the z direction be approximately 1 mm on average.

It is preferable that a depth D of the welded mark 170 fall within a range of from approximately 0.2 mm to 0.8 mm. It is more preferable that the depth D of the welded mark 170 fall within a range of from 0.5 mm to 0.7 mm.

### (4-5) Welding at Least at Two Portions

In the embodiment 1, welding which conforms to the above-mentioned (4-3) to (4-4) is performed at least at two portions. A plurality of portions where welding is performed are portions disposed separate from each other. When the number of portions where welding is performed is two, welding may be performed at positions in point symmetry about the rotary axis AX1 (see FIG. 5A).

When the number of portions where welding is performed is n (n being an integer of 2 or more), welding may be applied to the abutting profile 175 at positions of an equal angular pitch of 360°/n about the rotary axis AX1.

(4-6) Welding while Performing Pressing

Welding is performed while pressing the casing 120 to the base body 110 in the z direction. In other words, welding is performed in a state where a pressing force is applied between the second contact surface 122 and the first contact surface 113.

In applying the pressing force, in a case where the second engaging portion 125 and the first engaging portion 115 have the above-mentioned tapered shape, the first tapered portion of the first engaging portion (base body side) and the second tapered portion of the second engaging portion (casing side) may be engaged with each other thus generating a force in a direction that the casing is pressed in the z direction (base body side), and the casing 120 may be pressed to the base body 110 with such a pressing force in the z direction.

On the other hand, in a case where the second engaging portion 125 and the first engaging portion 115 have the above-mentioned non-tapered shape (straight shape), pressing may be indirectly applied from the second contact surface 122 to the first contact surface 113 by applying a force which presses the casing 120 to the base body 110 in the z direction using a jig or the like (not shown in the drawing).

By performing the above-mentioned steps ranging from the sub unit preparation step S10 to the welding step S40 in this order, the blood pump 100 according to the embodiment 1 can be obtained.

In addition to the above-mentioned steps ranging from the sub unit preparation step S10 to the welding step S40, as shown in FIG. 9, after the welding step S40 is performed, the blood pump disassembling step S50, the blood pump analyzing step S60, the casing refitting step S70, and the rewelding step S80 are performed in this order. Accordingly, it is possible to obtain the blood pump 100a which can be operable again while performing disassembling and analysis of the blood pump 100. Hereinafter, the respective steps are described sequentially.

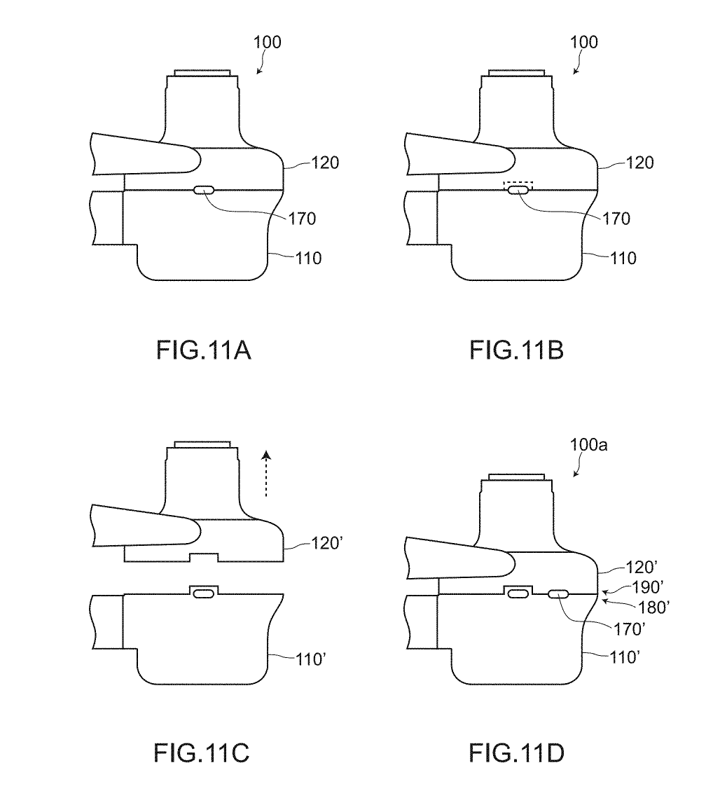

FIG. 11A to FIG. 11D are views for describing the blood pump disassembling step S50 and the rewelding step S80 in the embodiment 1.

### (5) Blood Pump Disassembling Step S50

The blood pump 100 is disassembled by performing at least a welded mark separation step S52 and a casing removing step S54 (see the blood pump disassembling step S50 shown in FIG. 9).

The welded mark separation step S52 is a step where a part of the casing 120 or/and the base body 110 which includes the welded mark 170 is separated from other parts of the casing 120 or/and the base body 110 which do not include the welded mark 170 from a state of the blood pump 100 where the welded mark 170 is formed on the blood pump 100 and the casing 120 is joined to the base body 110. To be more specific, for example, a boundary between a part of the casing 120 or/and the base body 110 which includes the welded mark 170 and the other parts which do not include the welded mark 170 is removed by cutting (cutting, fusing or the like) using a handy router, a laser or the like along a path indicated by a dotted line in FIG. 11B. With such an operation, a part of the casing 120 or/and the base body 110 which includes the welded mark 170 is separated from other parts which do not include the welded mark 170.

It is preferable that the above-mentioned removing by cutting be performed such that the neck portion 129 is included in the path. The reason for this is that among the casing intermediate portion 123, the second engaging portion 125, the casing edge portion 190 and the like, “neck portion 129” is a portion which has a smallest wall thickness and hence, the above-mentioned cutting by removing can be easily performed.

The casing removing step S54 is, as shown in FIG. 11C, a step for removing the casing 120 from the base body 110 by sliding the casing 120 in the −z direction with respect to the base body 110.

By performing the above-mentioned blood pump disassembling step S50 which includes the welded mark separation step S52 and the casing removing step S54, the blood pump 100 is disassembled. Accordingly, a casing 120′ and a base body 110′ form separate parts, and the inside of the casing 120′ and the inside of the base body 110′ are exposed to the outside.

### (6) Blood Pump Analyzing Step S60

Next, the blood pump is analyzed (see blood pump analyzing step S60 shown in FIG. 9).

In this embodiment, “analysis” includes the observation of a state of the blood pump regardless of the inside or outside of the blood pump, measurement of sizes of constitutional parts of the blood pump, an analysis of materials adhered to the inside of the blood pump and the like. However, “analysis” is not limited to these operations, and for example, working, readjustment and the like applied to the constitutional parts of the blood pump are also included in “analysis”.

### (7) Casing Refitting Step S70

Next, the casing 120′ is fitted on the base body 110′ again such that the second engaging portion 125 engages with the first engaging portion 115 by sliding the casing 120′ in the z direction with respect to the base body 110′ (casing refitting step S70 in FIG. 9).

As a detailed method for performing refitting, substantially the same method used in the casing fitting step S30 may be adopted.

### (8) Rewelding Step S80

Next, welding is performed again for connecting the casing edge portion 190′ and the base body stepped portion 180′ at least at two portions which are portions different from the portions where welding is performed in the welding step S40 and portions separated from each other while pressing the casing 120′ to the base body 110′ in the z direction (see rewelding step S80 shown in FIG. 9 and FIG. 11D).

As portions to be newly welded, different portions are selected such that such different portions avoid the welded marks 170 formed in the previously performed welding step S40. Such different portions are portions where a state of the casing edge portion 190′ (particularly, the casing outer end 193) and the base body stepped portion 180′ (particularly, the base body outer end 183) is not affected by the welding step S40 and hence, the rewelding step S80 can be newly performed in a stable manner. Such different portions are portions separated from each other, and at least two portions are selected as such different portions.

As a detailed method for performing rewelding, substantially the same method used in the welding step S40 may be adopted.

By performing the rewelding step S80, the casing 120′ and the base body 110′ can be strongly joined to each other again.

As described above, by performing the steps ranging from the blood pump disassembling step S50 to the rewelding step S80 in addition to the steps ranging from the sub unit preparation step S10 to the welding step S40, it is possible to obtain the blood pump 100a which is operable again while performing disassembling and analysis of the blood pump 100.

### 4. Advantageous Effects Obtained by Blood Pump 100 and Method of Manufacturing Blood Pump 100 According to Embodiment 1

(1) In the blood pump 100 according to the embodiment 1, the welded mark 170 which connects the casing edge portion 190 and the base body stepped portion 180 to each other is formed at least at two portions, and the second contact surface 122 of the casing 120 is pressed so as to be brought into contact with the first contact surface 113 of the base body 110.

That is, the casing 120 and the base body 110 are strongly joined to each other by welding at least at two portions in addition to joining by engagement between the first engaging portion 115 and the second engaging portion 125.

Accordingly, in the blood pump 100 according to the embodiment 1, a pressing force applied from the second contact surface 122 to the first contact surface 113 can be maintained.

Further, the blood pump 100 according to the embodiment 1 can enhance a casing peel-off strength compared to the conventional blood pump 800.

The blood pump 100 according to the embodiment 1 adopts the structure which requires no screws. It is unnecessary to ensure a width of the screw fastening margin 814 as in the case of the conventional blood pump 800 and hence, the blood pump 100 according to the embodiment 1 has a small diameter and a small volume compared to the conventional blood pump 800 and hence, the blood pump 100 according to the embodiment 1 becomes a miniaturized blood pump.

Further, in the blood pump 100 according to the embodiment 1, even when a thickness of the casing intermediate portion 123, the second engaging portion 125, or the casing edge portion 190 is set relatively small, strong joining is performed between the casing 120 and the base body 110 by partial welding at a final stage and hence, the blood pump 100 can maintain strong joining. That is, it is unnecessary to increase a thickness of the casing intermediate portion 123, the second engaging portion 125, or the casing edge portion 190 for generating a force generated by engagement for maintaining joining. Accordingly, also from this point of view, it is possible to provide the blood pump 100 according to the embodiment 1 in the form of a relatively miniaturized blood pump.

In this manner, accordingly to the embodiment 1, it is possible to provide the blood pump 100 which can increase a casing peel-off strength compared to the conventional blood pump 800 while maintaining a pressing force from the second contact surface 122 on a casing 120 side to the first contact surface 113 on abase body 110 side, and is smaller in size than the conventional blood pump 800.
(2) In the blood pump 100 according to the embodiment 1, the first tapered portion 116 inclined toward the −r direction as the first tapered portion 116 extends from the tapered outer end portion 117 in the z direction is formed on the first engaging portion 115 on a base body 110 side, and the second tapered portion 126 inclined toward the r direction as the second tapered portion 126 extends from the tapered inner end portion 127 in the −z direction on an inner wall of the casing 120 is formed on the second engaging portion 125 on a casing 120 side.

That is, due to the engagement between the first tapered portion 116 of the first engaging portion 115 and the second tapered portion 126 of the second engaging portion 125, a force in a direction which presses the casing 120 in the z direction (base body 110 side) is generated. With such a force which presses the casing 120 in the z direction, such a force can be further added as a portion of a pressing force applied to the contact surfaces, and such an additional force also contributes to the enhancement of a casing peel-off strength.

In this manner, according to the blood pump 100 of the embodiment 1, a casing peel-off strength can be further enhanced compared to the conventional blood pump 800.
(3) Assume a case where a blood pump is of a type where welding is applied to the whole circumference of the abutting profile 175 formed by the first profile 183a and the second profile 193a (whole circumference welding). In such a case, to disassemble the blood pump, it is necessary to remove by cutting a periphery of a welded mark over the whole circumference of the above-mentioned abutting profile 175.

On the other hand, in the blood pump 100 according to the embodiment 1, the welded mark 170 which connects the casing edge portion 190 and the base body stepped portion 180 to each other is disposed at least at two portions which are separated from each other (partial welding). Accordingly, at the time of disassembling the blood pump, it is sufficient to remove by cutting the periphery of the welded mark 170 only within a limited range and hence, the blood pump can be disassembled easily.

Further, in the blood pump 100 according to the embodiment 1, the blood pump 100 is assembled by partial welding as described above and hence, portions where the welded mark 170 does not exist remain in a wide range over the circumference of the abutting profile 175.

Accordingly, the welding for connecting the casing edge portion 190 and the base body stepped portion 180 can be performed again at portions different from the portions where welding is performed (the periphery of the welded mark 170) in the welding step S40 (rewelding step S80).

That is, in the embodiment 1, a partial welding mode is adopted. Accordingly, unlike the whole circumference welding, disassembling of the blood pump (blood pump disassembling step S50), reassembling (casing refitting step S70), rewelding (rewelding step S80) and the like can be easily performed.

Accordingly, a series of operations which includes disassembling of the blood pump 100, analysis (including not only the observation of a state, measurement of sizes, an analysis of adhered materials and the like but also working of constitutional parts and improvements such as readjustment), reassembling and rewelding, a reproduction test using the reconstructed blood pump 100a, confirmation of the advantageous effects of the improvements can be completed at a speedy cycle. Accordingly, the development and improvement activities for the enhancement of performance of the blood pump and the enhancement of reliability of the blood pump can be accelerated.
(4) According to the method of manufacturing the blood pump 100 according to the embodiment 1, the method includes in a following order: the casing fitting step S30 for fitting the casing 120 on the base body 110 such that the second engaging portion 125 engages with the first engaging portion 115; and the welding step S40 for performing welding so as to connect the casing edge portion 190 and the base body stepped portion 180 to each other while pressing the casing 120 to the base body 110 in the z direction.

In this embodiment, maintaining of a pressing force and ensuring of a casing peel-off strength are not obtained by only thread engagement or fitting between the casing and the base body. That is, in this embodiment, by performing the welding step S40 eventually in addition to the casing fitting step S30, final maintaining of a pressing force and final ensuring of a casing peel-off strength are established.

Accordingly, during a period from a point of time the casing fitting step S30 is finished to a point of time immediately before the welding step S40 starts, it is unnecessary to take into account maintaining of a pressing force and ensuring of a casing peel-off strength so much and hence, the removal (temporary disassembling) of the casing 120 from the base body 110 and fitting of the casing 120 on the base body 110 again are allowed in the midst of the period. Accordingly, it is possible to easily provide a step which requires temporary disassembling, for example, a step of inspecting an external appearance of the inside of the blood pump 100 during the period from the point of time the casing fitting step S30 is finished to the point of time immediately before the welding step S40 starts.

Further, according to the method of manufacturing the blood pump 100 of the embodiment 1, in the casing fitting step S30, fitting engagement between the casing 120 and the base body 110 is not performed by simply combining the casing 120 and the base body 110 each other, but is performed by making the second engaging portion 125 engage with the first engaging portion 115. Accordingly, there is no possibility that the casing 120 is removed from the base body 110 with a relatively small force.

Accordingly, it is unnecessary to use a jig for supporting the casing 120 and the base body 110 during the period from the point of time the casing fitting step S30 is finished to the point of time immediately before the welding step S40 starts.

### 5. Example of Blood Pump 100 According to Embodiment 1

An example in which the blood pump according to the present invention is carried out is described hereinafter.

A prototype of the blood pump 100 according to the embodiment 1 was manufactured and was rendered as the blood pump according to the example.

An impeller was adopted as the blood supply mechanism 130, a motor was adopted as the drive element 140, and pure titanium (F67, grade 2 stipulated in ASTM standard) was adopted as a material for forming the base body 110 and the casing 120. An outer diameter of the casing edge portion 190 (substantially also referred to as a diameter of the blood pump) when the blood pump 100 was viewed along the z direction was set to a value which falls within a range of from 35 mm to 60 mm. A thickness of the casing edge portion in the r direction was set to a value which falls within a range of from 0.2 mm to 0.7 mm.

The first engaging portion 115 and the second engaging portion 125 were respectively formed into a tapered shape.

In this example, a welded mark was formed at two portions positioned in point symmetry about the rotary axis AX1 from each other. A total length L of the welded marks 170 was set to a value which falls within a range of from 10 mm to 20 mm. A width Wz of the welded mark 170 in the z direction was set to a value which falls within a range of from 0.5 mm to 0.7 mm. A depth D of the welded mark 170 in the −r direction was set to a value which falls within a range of from 0.2 mm to 0.8 mm.

According to the blood pump of the example, a pressing force from the second contact surface 122 to the first contact surface 113 could sufficiently maintain a required specification. Further, a casing peel-off strength became a value which exceeds at least a range of from 1800 N to 4000 N and hence, it was confirmed that the casing peel-off strength was a sufficiently high value even when a metal fatigue limit strength brought about by fluctuation of a blood pressure is taken into consideration.

### Embodiment 2

FIG. 12 is a cross-sectional view for describing a main part of a blood pump 102 according to the embodiment 2. To be more specific, FIG. 12 is a cross-sectional view of the main part in a state where a casing 120″ is fitted on a base body 110″. FIG. 12 is a view which corresponds to FIG. 3B used in the embodiment 1. Parts having substantially the same configuration as corresponding parts of the embodiment 1 are given the same numerals.

The blood pump 102 according to the embodiment 2 has basically substantially the same configuration as the blood pump 100 according to the embodiment 1. However, the blood pump 102 according to the embodiment 2 differs from the blood pump 100 according to the embodiment 1 with respect to a shape of a first engaging portion and a shape of a second engaging portion.

That is, as shown in FIG. 12, in the blood pump 102 according to the embodiment 2, the first engaging portion 115′ and the second engaging portion 125′ are formed into a non-tapered shape (referred to as “straight shape” for the sake of convenience). In other words, when the blood pump 102 is cut along an xz plane which includes a rotary axis AX1, the first engaging portion 115′ and the second engaging portion 125′ are formed parallel to each other in the z direction. Further, an outer diameter of the first engaging portion 115′ and an inner diameter of the second engaging portion 125′ are set so as to take values extremely close to each other.

With such a configuration, in a casing fitting step S30, although the casing 120″ is fitted on the base body 110″ while receiving a slight resistance force, compared to the blood pump according to the embodiment 1 which includes the first engaging portion and the second engaging portion having a tapered shape, a large force is not necessary at the time of fitting the casing on the base body (casing fitting step S30, casing refitting step S70)/removal of the casing from the base body (casing removing step S54) and hence, such operations can be performed easily. Further, once the casing 120″ is fitted on the base body 110″, even when some external force is applied, the blood pump 102 can maintain a fitting state until a point of time immediately before a welding step S40/rewelding step S80 is performed.

The blood pump 102 according to the embodiment 2 has substantially the same configuration as the blood pump 100 according to the embodiment 1 except for the shape of the first engaging portion and the shape of the second engaging portion. Accordingly, the blood pump 102 according to the embodiment 2 directly acquires the corresponding advantageous effects found amongst all those advantageous effects which the blood pump 100 according to the embodiment 1 acquires.

### Embodiment 3

FIG. 13 is a cross-sectional view for describing a main part of a blood pump 103 according to the embodiment 3. To be more specific, FIG. 13 is a cross-sectional view of the main part in a state where a casing 120″ engages with a base body 110″, and is a view which corresponds to FIG. 12 used in the embodiment 2. Parts substantially equal to the constitutional elements of the embodiment 2 are given the same numerals.

The blood pump 103 according to the embodiment 3 has basically substantially the same configuration as the blood pump 100 according to the embodiment 1 and the blood pump 102 according to the embodiment 2. However, the blood pump 103 according to the embodiment 3 differs from the blood pump 100 according to the embodiment 1 and the blood pump 102 according to the embodiment 2 with respect to a point that the configuration relating to a packing 160 is added to the configuration of the blood pump 100 according to the embodiment 1 or the configuration of the blood pump 102 according to the embodiment 2. That is, as shown in FIG. 13, in the blood pump 103 according to the embodiment 3, a base body 110″ has a packing groove 119 in which the packing 160 is disposed at a position on a more −z direction side (upper side) of a first engaging portion 115″, a casing 120″ has an intermediate portion inner wall 124 at a position of a casing intermediate portion 123, and the packing 160 is disposed such that the packing 160 is sandwiched between the packing groove 119 and the intermediate portion inner wall 124.

In this manner, the blood pump 103 according to the embodiment 3 differs from the blood pump 100 according to the embodiment 1 and the blood pump 102 according to the embodiment 2 with respect to the point that the configuration relating to the packing 160 is added to the blood pump 103. However, according to the blood pump 103 of the embodiment 3, besides sealing obtained by a first contact surface 113 and a second contact surface 122, sealing is obtained by disposing the packing 160 based on the above-mentioned configuration, a leakage of blood to the outside of the blood pump can be blocked by double sealing. On the other hand, reversely, the intrusion of a bodily fluid or the like from the outside of the blood pump into the inside of the blood pump can be blocked by double sealing. Accordingly, the blood pump 103 according to the embodiment 3 becomes a blood pump having higher gastightness.

The packing is collapsed in the −r direction or in the r direction due to an elastic force of the casing intermediate portion and hence, there is no possibility that a diameter of the blood pump is increased.

When the blood pump 103 is viewed along the z direction, it is preferable that the packing 160 be disposed between the first and the second contact surfaces 113, 122 and a welded mark 170. This is because gastightness can be more effectively ensured by disposing the packing 160 in such a manner compared to a case where the welded mark 170 is disposed at a position close to the first contact surface 113 and the second contact surface 122. Further, at a place close to the first contact surface 113 and the second contact surface 122, a casing 120″ has a relatively large thickness and hence, the casing 120″ can collapse the packing 160 with certainty by its strong rigidity and hence, gastightness can be ensured with more certainty.

In the blood pump 103 according to the embodiment 3, when the blood pump 103 is viewed in a plan view along the z direction, it is preferable that the packing groove 119 formed on the base body 110″ overlap with an approximately horizontal surface 111 of the base body 110″, and the second contact surface 122 of the casing 120″ overlap with at least a portion of the packing 160.

According to the blood pump 103 having such a configuration, the packing 160 is assembled into the blood pump 103 in the form that at least a portion of a thickness of the packing 160 in the r direction is absorbed in a region of the approximately horizontal surface 111 and the second contact surface 122. Accordingly, the packing can be added without particularly increasing a diameter of the blood pump and hence, it is possible to provide a miniaturized blood pump.

In the description of the embodiment 3 made with reference to FIG. 13, the invention is disclosed where the configuration relating to the packing 160 is added to the blood pump 102 according to the embodiment 2. However, the blood pump 103 according to the embodiment 3 is not limited to such a configuration. That is, the configuration relating to the packing 160 may be added to the blood pump 100 according to the embodiment 1.

The blood pump 103 according to the embodiment 3 has substantially the same configuration as the blood pump 100 according to the embodiment 1 and the blood pump 102 according to the embodiment 2 except for the configuration relating to the packing 160. Accordingly, the blood pump 103 according to the embodiment 3 directly acquires the corresponding advantageous effects found amongst all advantageous effects which the blood pump 100 according to the embodiment 1 and the blood pump 102 according to the embodiment 2 acquire.

### Embodiment 4

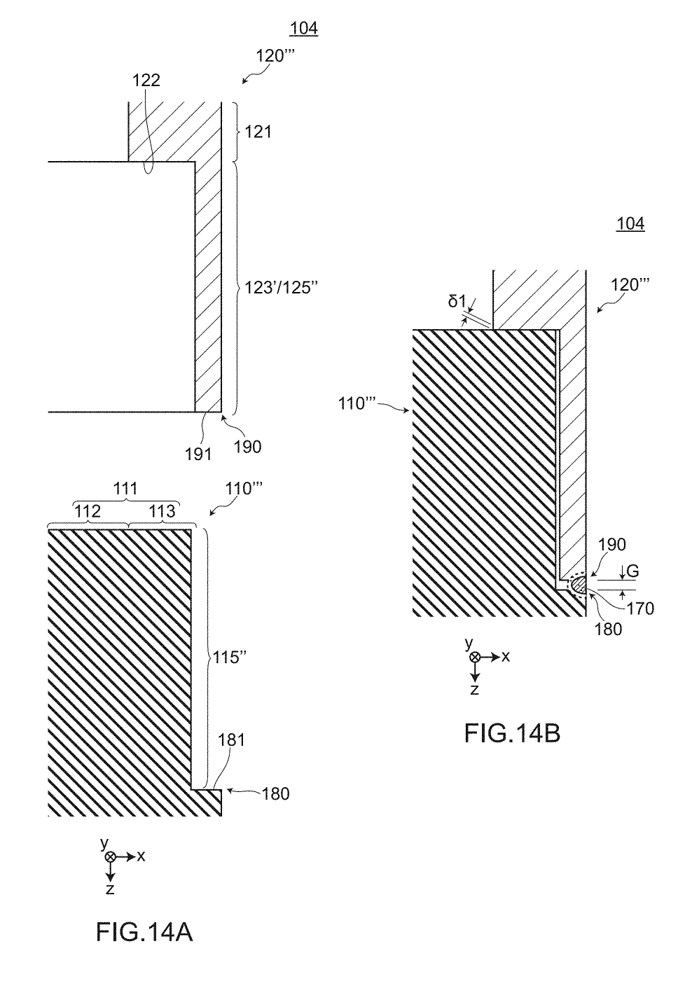

FIG. 14A and FIG. 14B are cross-sectional views for describing a main part of a blood pump 104 according to the embodiment 4. FIG. 14A is a view showing a state before a casing 120″′ is fitted on a base body 110″′, and FIG. 14B is a view showing a state after the casing 120″′ is fitted on and welded to the base body 110″′. Parts substantially equal to the constitutional elements of the embodiment 2 are given the same numerals.

The blood pump 104 according to the embodiment 4 has basically substantially the same configuration as the blood pumps 100, 102, 103 according to the embodiments 1 to 3. However, the blood pump 104 according to the embodiment 4 differs from the blood pumps 100, 102, 103 according to the embodiments 1 to 3 with respect to a point that a casing intermediate portion 123′ also functions as a second engaging portion 125″.

That is, as shown in FIG. 14A and FIG. 14B, in the blood pump 104 according to the embodiment 4, the casing intermediate portion 123′ and the second engaging portion 125″ are formed of the same portion, and such a portion is formed in an approximately straight shape together with an inner wall on a −r direction side and an outer wall on an r direction side. On the other hand, with respect to a base body 110″′, a shape of a first engaging portion 115″ within a region reaching an outer peripheral edge of a first contact surface 113 is formed in a shape which corresponds to a shape of an inner wall of the casing intermediate portion 123’ and the second engaging portion 125″.

In a state after the casing 120″′ is fitted on the base body 110″′ and before welding is performed, it is preferable that a slight gap G be formed between a base body stepped portion upper surface 181 and a casing edge portion lower surface 191. Further, it is preferable that a relationship of G»δ1 be established between the gap G and a gap δ1 (not shown in the drawing) between contact surfaces (a first contact surface 113 and a second contact surface 122).

The blood pump 104 according to the embodiment 4 has the configuration equivalent to the configurations of the blood pumps 100, 102, 103 according to the embodiments 1 to 3 and hence, the blood pump 104 according to the embodiment 4 has substantially the same configuration as the blood pumps 100, 102, 103 according to the embodiments 1 to 3 except for a point that the casing intermediate portion 123′ also functions as the second engaging portion 125″. Accordingly, the blood pump 104 according to the embodiment 4 directly acquires the corresponding advantageous effects found amongst all advantageous effects which the blood pumps 100, 102, 103 according to the embodiments 1 to 3 acquire.

### Embodiment 5

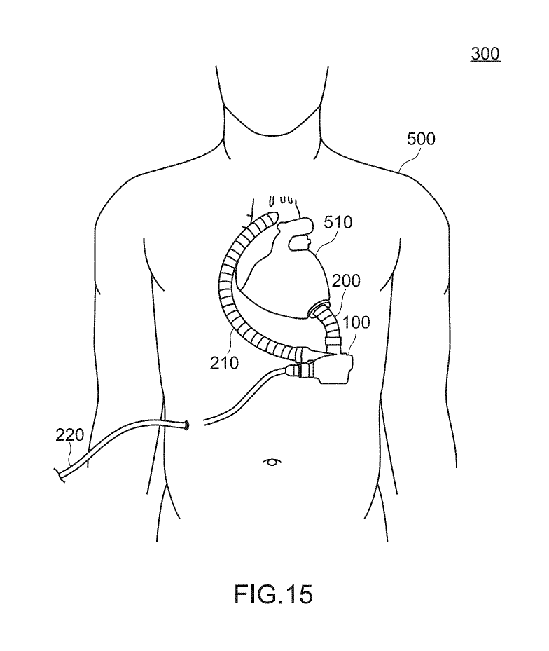

Next, an auxiliary artificial heart system 300 which uses the blood pump 100 is described as the embodiment 5 with reference to FIG. 15.

It is needless to say that the blood pump 100 according to the embodiment 1 may be mounted outside the body of a user. However, by using the blood pump 100 according to the embodiment 1 in the form of an auxiliary artificial heart by embedding the blood pump 100 in the inside of the body, the blood pump 100 can enjoy advantages from a view point of miniaturization and the reduction of weight.

When the blood pump is embedded in the inside of the body, the blood pump is embedded in a chest of a user (patient) having a limited thickness. Accordingly, with the use of the blood pump according to the present invention which has a small diameter and a small volume compared to conventional blood pumps while satisfying fundamental required specifications, the number of people who can use a blood pump can be increased. It is also possible to satisfy a demand in a medical field more preferably. In this specification, the expression that the blood pump is “embedded” in the body is used. However, besides such an expression, it is possible to use the expression that the blood pump is “implanted” in the body.

FIG. 15 is a schematic view for describing the auxiliary artificial heart system 300 according to the embodiment 5.

For example, as shown in FIG. 15, the auxiliary artificial heart system 300 includes: the blood pump 100 embedded in the body of a user; an artificial blood vessel 200 which connects the blood pump 100 and a left ventricle (not shown in the drawing) in the actual heart 510 of the user to each other; an artificial blood vessel 210 provided for returning blood from the blood pump 100 to the inside of the body of the user; a controller (not shown in the drawing) disposed outside the body of the user for controlling an operation of the blood pump 100; a cable 220 connecting the controller and the blood pump 100 to each other and the like.

As has been described heretofore, with the use of the blood pump 100 according to the embodiment 1 which has a small diameter and a small volume compared to the conventional blood pump 800 while satisfying required specifications, for example, it is possible to embed the blood pump 100 into the inside of the body of a person (patient) having a small physical build such as a child and hence, it is expected that the number of people who can use a blood pump is remarkably increased.

The blood pump used in the auxiliary artificial heart system 300 according to the embodiment 5 is not limited to the blood pump 100 according to the embodiment 1, and the blood pump 102 according to the embodiment 2 and the blood pump 103 according to the embodiment 3 can be also used in the auxiliary artificial heart system 300 according to the embodiment 5.

### [Provisional Test Example]

As a result of evaluation of a provisional test, it was confirmed that a casing peel-off strength S was increased substantially proportional to a total length L of the welded marks 170. The provisional test is described hereinafter.

### 1. Preparation of Specimens

As a specimen 1, a blood pump having the following configuration was prepared. In the blood pump, no welded mark 170 were formed, and a pressing force and a casing peel-off strength were maintained only by a first engaging portion 115 on which a first tapered portion 116 was formed and a second engaging portion 125 on which a second tapered portion 126 was formed. Further, a blood pump having substantially the same configuration as the blood pump 100 according to the embodiment 1 where a total length L of welded marks 170 was set to 2 mm (welded marks being disposed at two portions with a length of each welded mark set to 1 mm) was prepared as a specimen 2. A blood pump having substantially the same configuration as the blood pump 100 according to the embodiment 1 where a total length L of welded marks 170 was set to 4 mm (welded marks being disposed at two portions with a length of each welded mark set to 2 mm) was prepared as a specimen 3. A blood pump having substantially the same configuration as the blood pump 100 according to the embodiment 1 where a total length L of welded marks 170 was set to 10 mm (welded marks being disposed at two portions with a length of each welded mark set to 5 mm) was prepared as a specimen 4. A blood pump having substantially the same configuration as the blood pump 100 according to the embodiment 1 where a total length L of welded marks 170 was set to 20 mm (welded marks being disposed at two portions with a length of each welded mark set to 10 mm) was prepared as a specimen 5.

Assuming an inner diameter of a tapered inner end portion of the second tapered portion as ϕA, and an outer diameter of a tapered outer end portion of a first tapered portion as ϕB, the inner diameters ϕA and the outer diameters ϕB in the specimen 1 to the specimen 5 were set to values which fall within a range of from 35 mm to 60 mm.

### 2. Provisional Test Method

With respect to the specimen 1 to specimen 5, a casing 120 was pulled up in the −z direction (UP) in a state where a base body 110 was fixed, and a strength generated when the casing 120 was peeled off from the base body 110 was measured as a casing peel-off strength S.

### 3. Result of Provisional Test

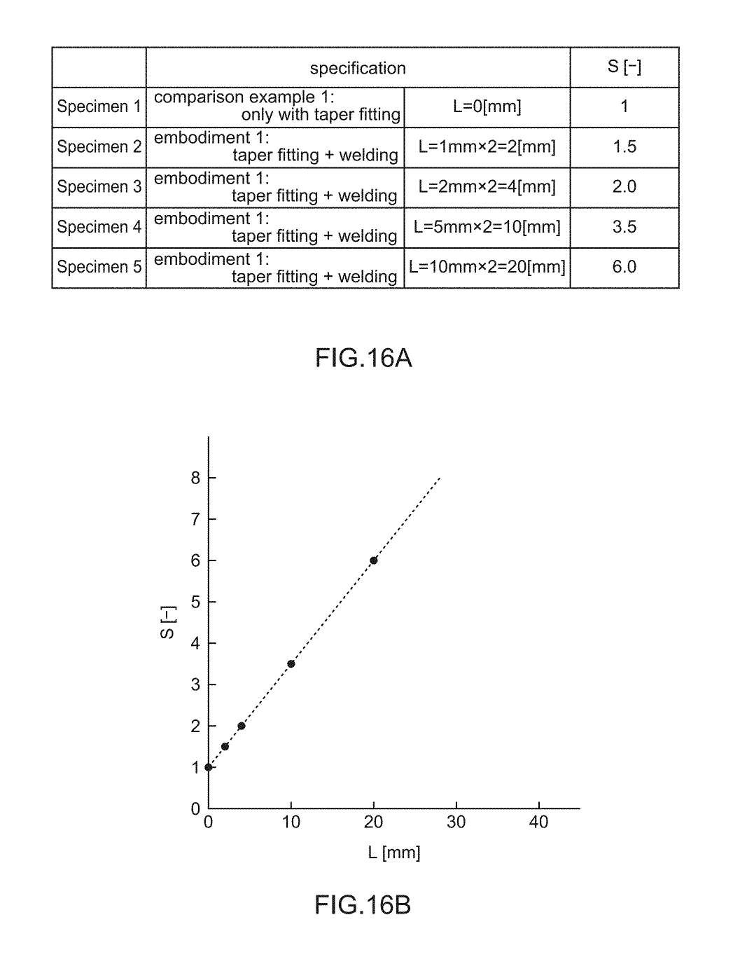

FIG. 16A and FIG. 16B are views for describing a result of evaluation of the blood pumps according to the provisional test examples. FIG. 16A is a table in which a relationship between a total length L of the welded marks 170 and a casing peel-off strength S was described with respect to the respective specimens in a collective manner. FIG. 16B is a graph obtained by plotting results of the provisional tests applied to the specimen 2 to the specimen 5, wherein a total length L of the welded marks 170 was taken on an axis of abscissas and a casing peel-off strength S was taken on an axis of ordinates, and the casing peel-off strength of the specimen 1 was set to 1.0. A dotted line is an auxiliary line.

It is confirmed from FIG. 16A and FIG. 16B that a casing peel-off strength S was increased approximately proportional to a total length L of the welded marks 170.

### [Test Example]

As a result of the evaluation of the test, it is confirmed that the blood pump 100 according to the embodiment 1 (specimen 5) and the blood pump 102 according to the embodiment 2 (specimen 6) could increase a casing peel-off strength S while maintaining a pressing force compared to the conventional blood pump (comparison example 2: specimen 7), and the blood pump 100 according to the embodiment 1 (specimen 5) and the blood pump 102 according to the embodiment 2 (specimen 6) became blood pumps smaller than the conventional blood pumps in size. The test example is described hereinafter.

### 1. Preparation of Specimens

The specimen 5 formed in the above-mentioned provisional test example was prepared. The specimen 5 has substantially the same configuration as the blood pump 100 according to the embodiment 1. A blood pump having substantially the same configuration as the blood pump 102 according to the embodiment 2 where a total length L of welded marks 170 was set to 20 mm (welded marks being disposed at two portions with a length of each welded mark set to 10 mm) was prepared as a specimen 6. The specimen 1 formed in the above-mentioned provisional test example was prepared as a comparison example 1. A blood pump formed by joining a casing and a base body using screws substantially equal to the conventional blood pump 800 and forms a comparison example 2 was prepared as a specimen 7.

### 2. Test Method

A pressing force, a casing peel-off strength S measured by a method substantially equal to the method used in the measurement in the provisional test, an outer diameter of a portion in the vicinity of a casing edge portion 190 (considered approximately equal to a diameter of a casing outer end 193), a volume ratio of the blood pump, and a weight ratio of the blood pump were respectively evaluated with respect to the specimens 5, 6, 1, and 7.

### 3. Result of Test

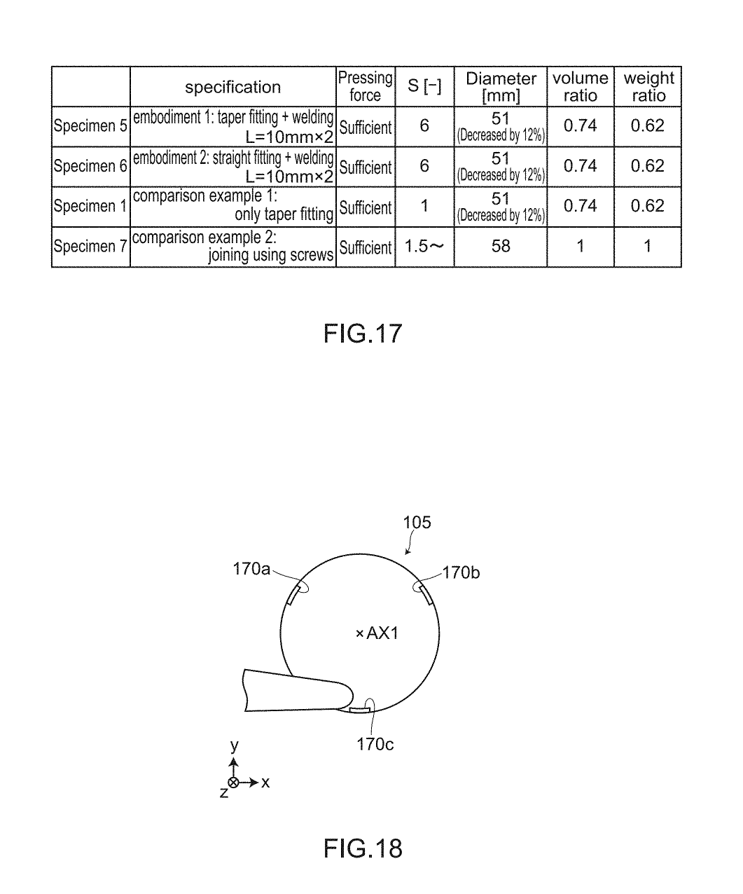

FIG. 17 is a view for describing a result of evaluation of the blood pumps according to the test example.

As shown in FIG. 17, to study the result of evaluation of the specimen 5 and the specimen 6, a casing peel-off strengths S of the specimen 5 and the specimen 6 were 4 times or less as large as a casing peel-off strength S of the specimen 7 and the casing peel-off strengths are increased. Further, volume ratios of the specimen 5 and the specimen 6 were 0.74 times as large as a volume ratio of the specimen 7, and weight ratios of the specimen 5 and the specimen 6 were 0.62 times as large as a weight ratio of the specimen 7.

Accordingly, it was confirmed that the blood pump 100 according to the embodiment 1 (specimen 5) and the blood pump 102 according to the embodiment 2 (specimen 6) could increase a casing peel-off strength S while maintaining a pressing force compared to the conventional blood pump (comparison example 2: specimen 7), and the blood pump 100 according to the embodiment 1 (specimen 5) and the blood pump 102 according to the embodiment 2 (specimen 6) became smaller than the conventional blood pump in size.

Although the present invention has been described based on the respective embodiments, the present invention is not limited to the above-mentioned respective embodiments, and the present invention can be carried out in various modes without departing from the gist of the present invention. For example, the following modifications are also conceivable.
(1) The numbers, the materials, the shapes, the positions, the sizes and the like of the constitutional elements described in the above-mentioned respective embodiments are provided only for an exemplifying purpose, and these can be changed within ranges where advantageous effects of the present invention are not impaired.
(2) FIG. 18 is a view for describing a blood pump 105 according to a modification 1, and is a plan view when the blood pump 105 is viewed along the z direction. Parts substantially equal to the constitutional elements of the embodiment 1 are given the same numerals.

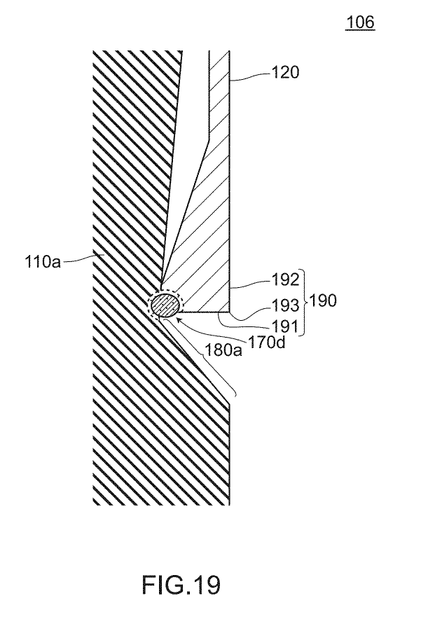

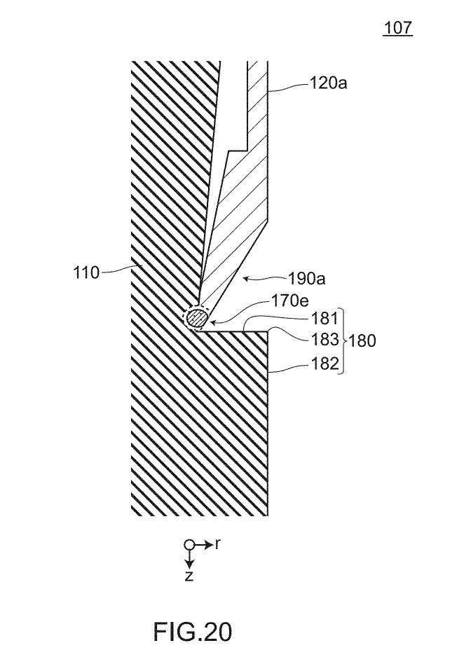

In the above-mentioned respective embodiments, the description has been made with respect to the example where the number of portions where the welded mark 170 is disposed is two. However, the present invention is not limited to such an example. For example, as shown in FIG. 18, the configuration may be adopted where welded marks 170a, 170b, 170c and the like are disposed at three or more portions (modification 1).
(3) FIG. 19 is a view for describing a blood pump 106 according to a modification 2. To be more specific, FIG. 19 is a cross-sectional view of a main part in a state where a casing 120 is fitted on a base body 110a. FIG. 20 is a view for describing a blood pump 107 according to a modification 3. To be more specific, FIG. 20 is a cross-sectional view of a main part in a state where a casing 120a is fitted on a base body 110. In these modifications 1 and 2, parts substantially equal to the constitutional elements of the embodiment 1 are given the same numerals.

In the above-mentioned respective embodiments, the description has been made with respect to the case where the welded mark 170 which connects the casing edge portion 190 and the base body stepped portion 180 is the welded mark 170 formed by applying welding between the first profile 183a of the base body outer end 183 and the second profile 193a of the casing outer end 193 as a typical example. However, the present invention is not limited to such a welded mark. That is, it is not always necessary to perform welding between the base body outer end 183 and the casing outer end 193.

For example, as shown in FIG. 19, a base body stepped portion 180a may be formed in an oblique tapered shape, and a welded mark 170d may be formed by applying welding to a portion of the base body stepped portion 180a on a −r direction side and a portion of the casing edge portion 190 on a −r direction side (modification 2).

Further, for example, as shown in FIG. 20, an outer side of a casing edge portion 190 may be formed in an oblique tapered shape, and a welded mark 170e may be formed by applying welding to a portion of a base body stepped portion 180 on a −r direction side (modification 3).
(4) In the above-mentioned respective embodiments, the description has been made with respect to the example where a welded mark is formed between the first profile 183a of the base body outer end 183 formed so as to surround the rotary axis AX1 of the blood supply mechanism 130 and the second profile 193a of the casing outer end 193 formed so as to surround the rotary axis AX1 of the blood supply mechanism 130, and the first profile 183a and the second profile 193a form a continuous circle respectively. However, the present invention is not limited to such a case.

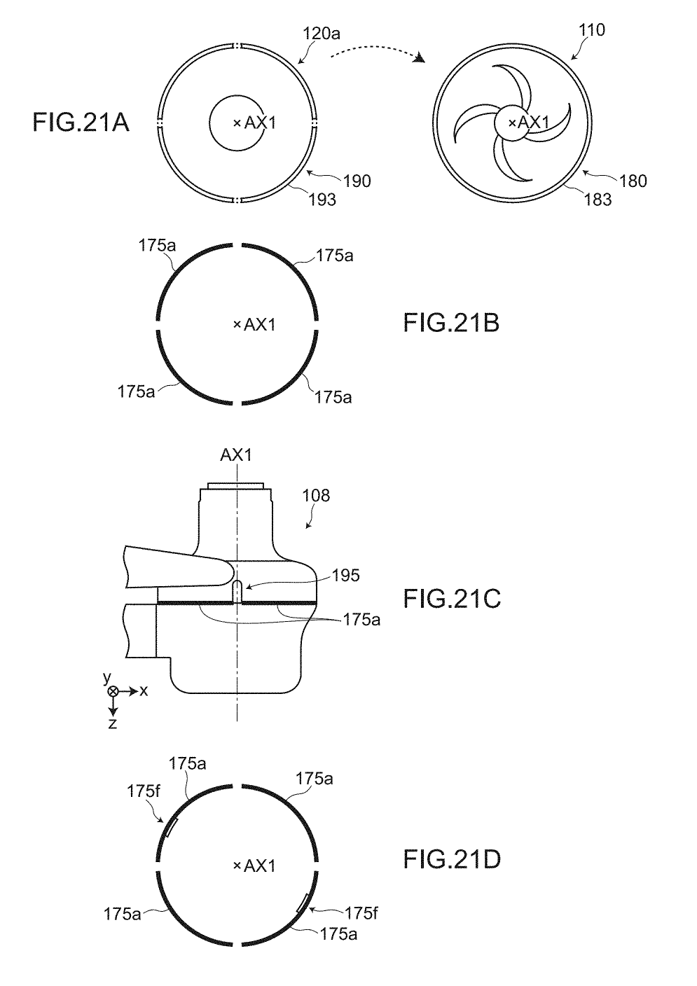

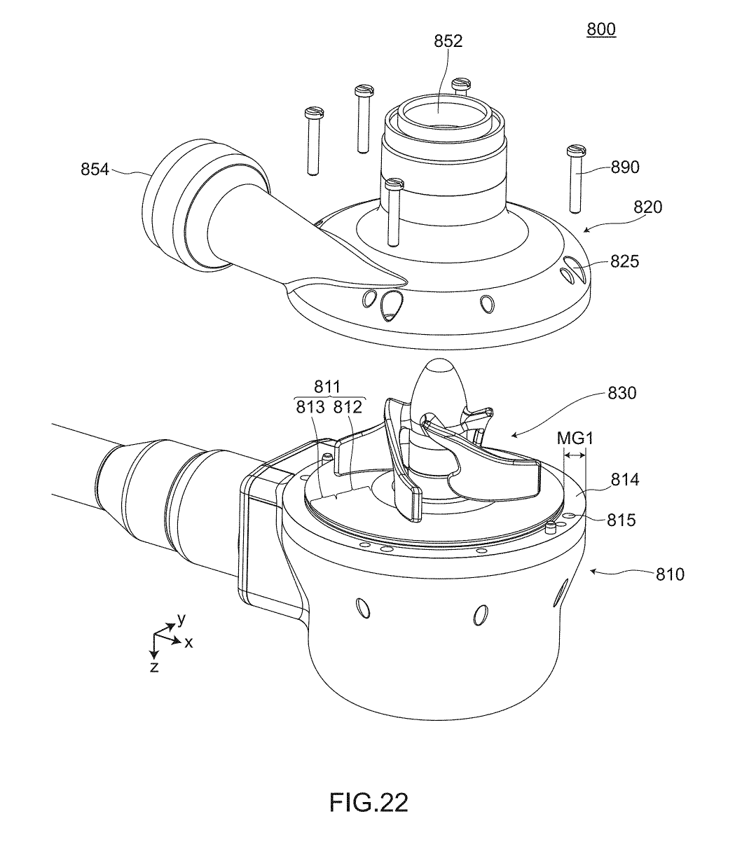

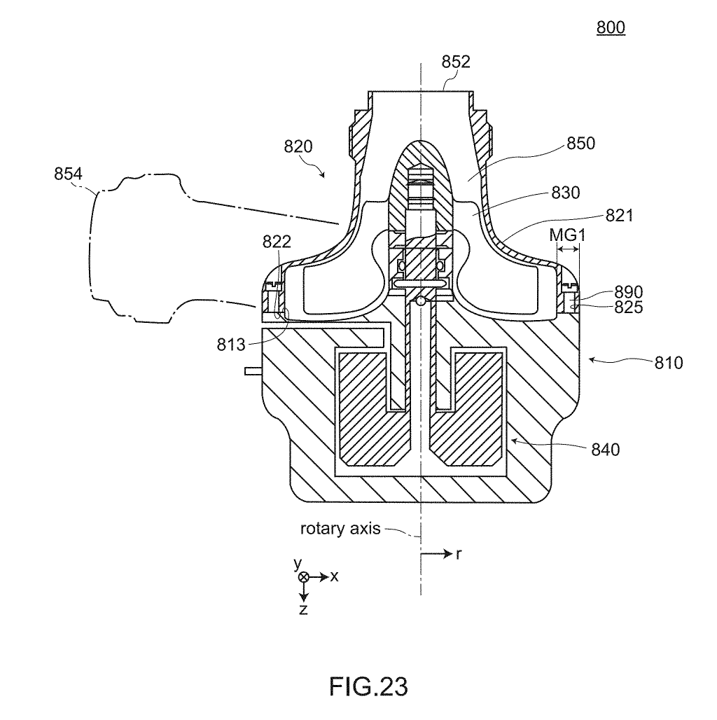

FIG. 21A to FIG. 21D are views for describing a blood pump 108 according to a modification 4. In the modification 4, parts substantially equal to the constitutional elements of the embodiment 1 are given the same numerals.

For example, as shown in FIG. 21A to FIG. 21D, “slits 195” may be formed in a casing outer end 193, and a second profile 193a may be formed by the discontinuous circular casing outer end 193 having the slits, where the casing outer end 193 is formed so as to surround a rotary axis AX1 of a blood supply mechanism 130 (modification 4).

1. A blood pump comprising:
a base body;
a casing fitted on the base body;
a blood supply mechanism housed in a pump chamber surrounded by the base body and the casing; and
a drive element mounted on the base body for supplying energy to the blood supply mechanism, wherein
the blood supply mechanism is configured to allow blood to flow into the pump chamber and to flow out from the pump chamber so as to supply blood into the inside of a body of a user, wherein
assuming a direction that the casing is fitted on the base body by sliding as a z direction, a direction perpendicular to the z direction as an x direction, a direction perpendicular to the z direction and the x direction respectively as a y direction, a direction directed from a center portion of the base body to the outside of the base body when an xy plane is viewed in a plan view along the z direction as an r direction, a direction opposite to the z direction as a −z direction, and a direction opposite to the r direction as a −r direction,
the base body has:
an approximately horizontal surface formed on a −z direction side, and including a blood contact surface which faces the pump chamber and a first contact surface which is brought into contact with the casing;
a first engaging portion formed on an r direction side of the base body; and
a base body stepped portion formed on a z direction side of the first engaging portion,
the casing has:
a casing body having a second contact surface at a position which corresponds to the first contact surface;
a second engaging portion formed at a position of an edge side of the casing with respect to the casing body;
a casing intermediate portion positioned between the casing body and the second engaging portion; and
a casing edge portion positioned on a side opposite to a side of the casing intermediate portion with respect to the second engaging portion,
the base body stepped portion is disposed at a position which corresponds to the casing edge portion in a state where the casing is fitted on the base body, and
the blood pump is configured such that the second engaging portion of the casing engages with the first engaging portion of the base body, a welded mark which connects the casing edge portion and the base body stepped portion to each other is formed at least at two portions which are separated from each other, and the second contact surface of the casing is pressed so as to be brought into contact with the first contact surface of the base body.

2. The blood pump according to claim 1, wherein
on the casing edge portion, a casing edge portion lower surface which is a surface of an edge of the casing on a z direction side, a casing edge portion outer surface which is a surface of the edge of the casing on an r direction side, and a casing outer end which is a corner formed by the casing edge portion lower surface and the casing edge portion outer surface are formed,
on the base body stepped portion, a base body stepped portion upper surface which is formed in an abutting manner with the casing edge portion lower surface in a state where the casing is fitted on the base body, a base body stepped portion outer surface which is a surface of the base body stepped portion on an r direction side, and a base body outer end which is a corner formed by the base body stepped portion upper surface and the base body stepped portion outer surface are formed, and
in the blood pump, the welded mark which connects the casing edge portion and the base body stepped potion to each other is formed at least at two portions which are separated from each other between a first profile of the base body outer end formed so as to surround a rotary axis of the blood supply mechanism and a second profile of the casing outer end formed so as to surround the rotary axis of the blood supply mechanism.

3. The blood pump according to claim 1, wherein
a first tapered portion inclined toward the −r direction as the first tapered portion extends from a tapered outer end portion in the z direction is formed on the first engaging portion, and
a second tapered portion inclined toward the r direction as the second tapered portion extends from a tapered inner end portion in the −z direction on an inner wall of the casing is formed on the second engaging portion.

4. The blood pump according to claim 1, wherein
assuming an inner diameter of the tapered inner end portion of the second tapered portion of the casing as ϕA and an outer diameter of the tapered outer end portion of the first tapered portion of the base body as ϕB before the casing is fitted on the base body, a relationship of ϕA<ϕB is established, and
the casing is fitted on the base body in a state that a position of the tapered inner end portion of the second tapered portion is disposed at a portion of the first tapered portion shifted in a z direction side from the tapered outer end portion, the portion being a portion of the first tapered portion away a first tapered terminal end portion on a side opposite to the tapered outer end portion.

5. The blood pump according to claim 1, wherein
assuming an angle made by a profile of an inclined surface of the first tapered portion and the z direction as ϕ1 and an angle made by a profile of an inclined surface of the second tapered portion and the z direction as ϕ2, a relationship of 0°≤ϕ1≤ϕ2 is established.

6. The blood pump according to claim 1, wherein
assuming a thickness of the casing body in a direction perpendicular to a tangent plane on an outer side of the casing body as t1, a thickness of the casing intermediate portion in a direction perpendicular to a tangent plane of an outer side of the casing intermediate portion as t2, and a thickness of the second engaging portion in a direction perpendicular to a tangent plane of an outer side of the second engaging portion as t3, a relationship of t1>t2>t3 is established, and
with respect to an inclined surface which forms the second tapered portion of the second engaging portion, on a second tapered terminal end portion side which is a side opposite to the tapered inner end portion, the casing has a smallest thickness.

7. The blood pump according to claim 1, wherein
a width of the welded mark in the z direction falls within a range of from 0.3 mm to 2.0 mm, and
a total length of the welded marks falls within a range of from 2 mm to 40 mm when the welded marks are viewed along the z direction.

8. The blood pump according to claim 7, wherein
the total length of the welded marks is 5 mm or more when the welded marks are viewed along the z direction.

9. The blood pump according to claim 7, wherein
the total length of the welded marks is 30 mm or less when the welded marks are viewed along the z direction.

10. The blood pump according to claim 2, wherein
an underfill is formed on the welded mark, and
a gap between a deepest portion of the underfill and the casing edge portion outer surface or the base body stepped portion outer surface falls within a range of from 0.05 mm to 0.3 mm.

11. The blood pump according to claim 1, wherein
assuming a thickness of the casing intermediate portion in a direction perpendicular to the tangent plane of the outer side of the casing intermediate portion which is a thickness of a portion of the casing having a smallest thickness as t2′, and
assuming a thickness of the second engaging portion in a direction perpendicular to the tangent plane of the outer side of the second engaging portion as t3, a relationship of t2′<t3 is established.

12. The blood pump according to claim 1, wherein
the base body has a packing groove in which a packing is disposed at a position on a more −z direction side of the first engaging portion,
the casing has an intermediate portion inner wall at a position of the casing intermediate portion, and
the packing is disposed such that the packing is sandwiched between the packing groove and the intermediate portion inner wall.

13. A method of manufacturing a blood pump for manufacturing the blood pump described in claim 1, the method comprising in a following order:
a sub unit preparation step for preparing the base body, the casing, and the blood supply mechanism;
a blood supply mechanism mounting step for mounting the blood supply mechanism on the base body;
a casing fitting step for fitting the casing on the base body such that the second engaging portion engages with the first engaging portion by sliding of the casing in the z direction with respect to the base body; and
a welding step for performing welding so as to connect the casing edge portion and the base body stepped portion to each other at least at two portions which are separated from each other while pressing the casing to the base body in the z direction.

14. The method of manufacturing a blood pump according to claim 13 further comprising in a following order after the welding step:
a blood pump disassembling step including: a welded mark separation step for separating a portion of the casing or/and the base body including a welded mark from other portions which include no welded mark; and a casing removing step for removing the casing from the base body by sliding the casing in the −z direction with respect to the base body;
a blood pump analyzing step for analyzing the blood pump;
a casing refitting step for fitting the casing on the base body again such that the second engaging portion engages with the first engaging portion by sliding the casing in the z direction with respect to the base body; and
rewelding step for performing again welding for connecting the casing edge portion and the base body stepped portion to each other at least at two portions which are separated from each other and are portions different from portions where welding is performed in the welding step while pressing the casing to the base body in the z direction.
